(19) United States
(12) Patent Application Publication
(10) Pub. No.: US 2025/0009225 A1
(43) Pub. Date: Jan. 9, 2025

(54) PATIENT BEHAVIOR EVALUATION USING VISION SCREENING DEVICE

(71) Applicant: Welch Allyn, Inc., Skaneateles Falls, NY (US)

(72) Inventors: Stacie Lynn Brough, Syracuse, NY (US); David L. Kellner, Baldwinsville, NY (US)

(73) Assignee: Welch Allyn, Inc., Skaneateles Falls, NY (US)

(21) Appl. No.: 18/766,389

(22) Filed: Jul. 8, 2024

Related U.S. Application Data
(60) Provisional application No. 63/525,610, filed on Jul. 7, 2023.

(51) Int. Cl. A61B 3/14; A61B 3/12; G16H 10/60; G16H 20/00; G16H 50/30

(52) U.S. Cl. CPC A61B 3/14; A61B 3/12; G16H 10/60; G16H 20/00; G16H 50/30

(57) ABSTRACT

A vision screening device for administering vision screening tests to a patient, to determine the presence of diseases and/or abnormalities in the eye(s) of the patient, is described herein. The vision screening device may include associated methods and systems configured to perform the operations of the vision screening tests. The device may include a radiation source configured to generate near-infrared (NIR) radiation, a sensor configured to capture a grayscale image representing the radiation reflected by the eye(s) of the patient, a white light source, and a camera configured to capture a color image of the eye of the patient. The device may also be configured to generate a behavior likelihood score for a patient based on the captured data, the score indicative of a likelihood of particular behavior (e.g., violence, outbursts, withdrawal symptoms, etc.). The device may then recommend action to prepare for such behavior, if necessary.

### CROSS-REFERENCE TO RELATED APPLICATIONS

This application claims priority to U.S. provisional patent application Ser. No. 63/525,610, filed Jul. 7, 2023, which is incorporated herein by reference for all purposes.

### TECHNICAL FIELD

This application is directed to medical equipment. In particular, this application is directed to a vision screening device, and associated systems and methods, for detection and assessment of diseases and disorders of the eye and use of the vision screening device for evaluating and predicting patient behavior.

### BACKGROUND

Vision screening typically includes screening for diseases of the eye. Such screening may include, for example, a transillumination test such as the Brückner red reflex test. During the red reflex test, the clinician illuminates the eye of the patient with visible light using an ophthalmoscope, and examines the color and other characteristics of the light reflected back by the choroid and the retinal surfaces of the eye. Various diseases and abnormalities of the eyes can be detected using this test, such as corneal or media opacities, cataracts, and retinal abnormalities including tumors and retinoblastoma. Vision screening for diseases is recommended for all age groups. For example, newborns may be screened for congenital eye diseases, while older adults may be screened for the onset of age-related degenerative diseases such as cataracts and retinal diseases. The presence of foreign objects in the eye may also be detected using vision screening under visible light.

In addition, vision screening typically also includes one or more tests to determine various deficiencies associated with the patient's eyes. Such vision tests may include, for example, refractive error tests, accommodation tests, visual acuity tests, color vision screening and the like. Some of the vision screening tests require the use of infrared or near-infrared imaging, while other tests may require imaging under visible light, and/or a display screen to show content to the patient. However, ophthalmic testing devices such as a phoropter, autorefractor and photo-refractors, may only provide the capability to perform a limited range of tests. It would be advantageous to be able to screen for most vision problems and diseases using a single integrated device.

The various examples of the present disclosure are directed toward overcoming one or more of the deficiencies noted above.

### SUMMARY

In an example of the present disclosure, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method that includes illuminating, using a vision screening device, an eye of a patient during a first period of time. The method further includes capturing, using the vision screening device, image data of the eye during the first period of time. The method also includes determining a characteristic of the eye of the patient during the first period of time based on the image data and determining a likelihood score based on the characteristic of the eye, the likelihood score indicative of a probability associated with one or more condition predictions or one or more behavior predictions for the patient. The method further includes generating, based at least in part on the likelihood score, an output indicative of a behavior predicted for the patient. Other embodiments of this aspect include corresponding computer systems, apparatus, and computer programs recorded on one or more computer storage devices, each configured to perform the actions of the methods.

### BRIEF DESCRIPTION OF THE DRAWINGS

Features of the present disclosure, its nature, and various advantages, may be more apparent upon consideration of the following detailed description, taken in conjunction with the accompanying drawings.

FIG. 1 illustrates an example system for performing patient behavior evaluations using a vision screening system.

FIG. 2 illustrates an example vision screening device and vision screening system of the present disclosure.

FIG. 3 illustrates an example vision screening device and computing system of the present disclosure.

FIG. 4 illustrates an example vision screening device of the present disclosure.

FIGS. 5A-5D illustrate example characteristics that may be used by a vision screening device to evaluate behavioral predictions, according to examples of the present disclosure.

FIG. 6 provides a first flow diagram illustrating an example method of the present disclosure.

FIG. 7 illustrates an example device configured to enable and/or perform some or all of the functionality discussed herein.

In the figures, the left-most digit(s) of a reference number identifies the figure in which the reference number first appears. The use of the same reference numbers in different figures indicates similar or identical items or features. The drawings are not to scale.

### DETAILED DESCRIPTION

The present disclosure is directed to, in part, a vision screening device, and corresponding methods. Such an example vision screening device may be configured to perform one or more vision screening tests on a patient and to output the results of the vision screening test(s) to an operator of the device, such as a clinician or a physician's assistant. Specifically, the present disclosure is directed to devices and methods for screening patients for impairment, such as for drug abuse, as part of an initial physical assessment and evaluating and/or predicting a likelihood of particular behavior from the patient based on the evaluation. For example, the vision screening device may capture one or more images of the eye illuminated by radiation of different wavelength ranges of electromagnetic spectrum (e.g., infrared, near-infrared, and visible light). The device may determine, based on analysis of the captured images, one or more diseases and/or abnormalities of the eyes, such as cataracts, tumors, ametropia, foreign body in the eye, corneal abrasions, retinal detachment or lesions, congenital conditions and the like, associated with one or both eyes of the patient. The device may further be used to detect if a patient is impaired and may be used to screen or identify patients that may be at a higher risk for potentially violent behavior or other predicted behavior, based on the captured images.

The device may also generate visualizations of the captured images for displaying to the clinician or the operator of the vision screening device to assist the clinician or the operator in determining a diagnosis. As such, the methods described herein may provide an automated diagnosis based on the analysis of images captured by the vision screening device. The methods described herein may also provide an automated recommendation based on and/or indicative of such a diagnosis.

For example, caregiver violence is a known problem in emergency departments, many as a result of drug abuse or overdose reversals, with behavior such as precipitated withdrawal symptoms, anger, and aggression. Many major drugs and substances, such as cocaine, marijuana, amphetamine, phencyclidine, heroin, and alcohol, may produce eye signs that can be detected by an eye test. The characteristics that may be evaluated include ptosis, abnormal pupil size, nonreactivity of the pupil to a light challenge, nystagmus, non-convergence, hippus, redness or swelling at or around the eyes, and other such visibly detectable characteristics. In some examples, the vision screening device may detect conditions indicative of substance abuse, impairment, central nervous system issues, epilepsy, tumors, and other such conditions. In some examples, nystagmus may refer to repetitive, uncontrolled movements of the eyes. Nystagmus may be identifiable based on eye jitters or sudden jumps by the eyes as they track an item. In some examples, nystagmus may be identified when the eyes exhibit a linear jump greater than ten percent of a moving distance to track an item. Hippus may refer to a restless mobility of the pupil, a tendency for the pupil size to fluctuate when it should otherwise be stable. For example, a pupil of an individual may pulsate or change in diameter over a short period of time. The vision screening device described herein provides for a reliable way to detect and evaluate and/or predict patients that are using substances that may be a predictor of unstable behavior or likelihood of needing an overdose reversal that would anticipate the need for additional staff, security, or other department resources to protect caregivers from a patient's violent outburst.

Therefore, based at least in part on the analysis of the captured images from the vision screening device, the device (and/or a computing device associated with the device) may generate an output including a recommendation, prediction, evaluation, or other indication of the status of the patient and/or a prediction of certain behaviors such as outbursts, flight risk, violent behavior, etc. For example, the device may detect if a patient is impaired due to drug use and indicate a violence risk score based on the captured images. Notably, the vision screening device may provide such indications earlier than a blood test may return a result indicative of such impairment or altered status and therefore the caregivers may be prepared to allocate resources as necessary before an incident occurs.

The vision screening device may be used as part of the routine physical assessment at triage on a patient when they enter an emergency department or other caregiving location. The vision screening device may automate an eye test typically performed by physicians and output an indication to caregivers with a more limited scope of practice. For instance, the output may include a risk score associated with a warning of violence propensity. In some examples, after the vision screening device is used to perform the test, the captured images or other such data gathered by the vision screening device can be used to determine one or more behavior scores indicative of likelihoods associated with particular behavior. The determination can be made on-board the vision screening device and/or by a computing device communicably coupled (e.g., over a network) with the vision screening device and may include the use of a machine learning, artificial intelligence, or other algorithm to analyze the captured images. The algorithms or other such techniques may be trained using eye exam data tied to patient data that documents particular behaviors such as violent outbursts.

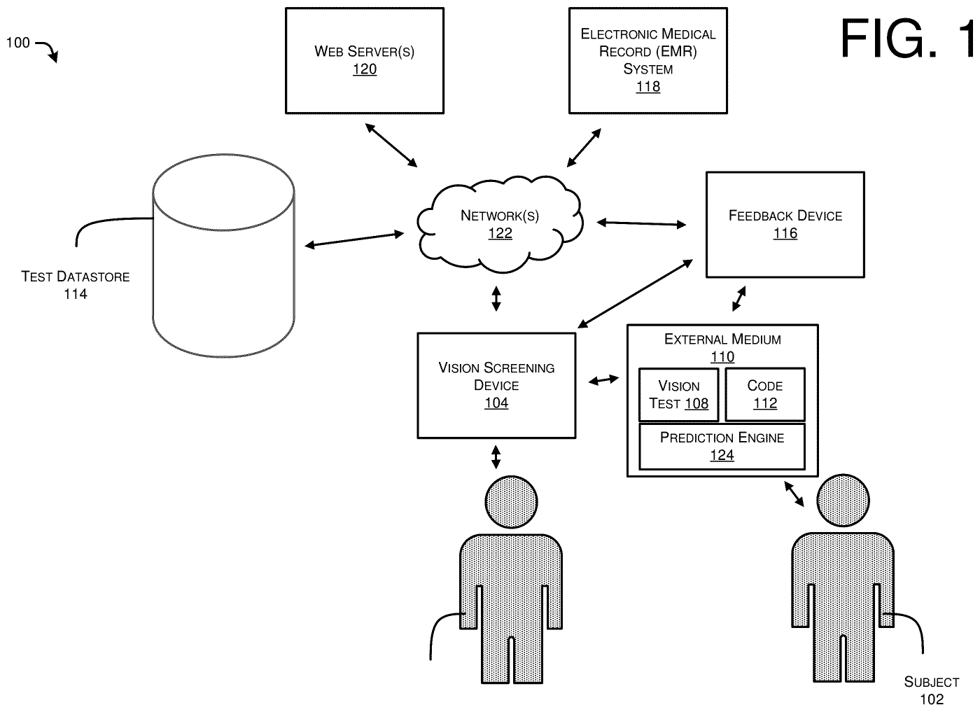

As will be described with respect to at least FIG. 1, an example vision screening device associated with screening for diseases and abnormalities of the eyes may include components for capturing images of the eye(s) of the patient under near-infrared as well as visible light radiation. The device may include components for controlling emission of near-infrared and visible light radiation and the corresponding capture of the reflected radiation from the eye(s) during the screening. In examples, near-infrared images may be captured before initiating the capture of visible light images, so that pupils of the eyes(s) of the patient do not constrict, or accommodate, during the screening in response to visible light, and the screening may be completed without the need for dilation of the eyes. The device may further include components for analyzing the captured images to determine disease conditions and/or abnormalities in the eye(s) of the patient, and components for determining and reporting of output(s) indicating the disease conditions and/or abnormalities detected during the screening.

Additional details pertaining to the above-mentioned devices and techniques are described below with reference to FIGS. 1-8. It is to be appreciated that while these figures describe devices and systems that may utilize the claimed methods, the methods, processes, functions, operations, and/or techniques described herein may apply equally to other devices, systems, and the like.

FIG. 1 illustrates a system 100 for performing patient behavior evaluations using a vision screening system. In various implementations, at least one eye of a subject 102 is screened for at least one ocular condition. As used herein, the terms “ocular condition,” “ophthalmic condition,” “condition,” and their equivalents, can refer to a pathologic state of an individual that is associated with a state of at least one eye of the individual. Some ocular conditions, for example, are pathological conditions of the eye itself, such as amblyopia, myopia, hyperopia, astigmatism, cataract, retinopathy, color vision deficiency, macular degeneration, and so on. Some ocular conditions are pathological conditions of other areas of the body, but can be identified based on the appearance and/or performance of the eye. Other examples of ocular conditions include concussion, learning disorders (e.g., dyslexia), some cancers, and so on.

During the screening for the ocular condition, or in place of the screening, the subject 102 may be evaluated for predicted behavior, as described herein. For example, the subject 102 may be screened as part of an entrance triage at a care facility. The subject 102 may be screened such that a risk score for a particular behavior (flight risk, violence, outbursts, withdrawals, etc.) may be determined that can be used by the care facility to distribute resources and treat the subject 102 appropriately. The screening for the eye test may allow for evaluation of responsiveness, motion, or reactivity of pupils, or other such pupil and/or eye characteristics that may be evaluated include ptosis, abnormal pupil size, nonreactivity of the pupil to a light challenge, nystagmus, non-convergence, redness or swelling at or around the eyes, and other such visibly detectable characteristics. The vision screening device described herein provides for a reliable way to detect and evaluate and/or predict patients that are using substances that may be a predictor of unstable behavior or likelihood of needing an overdose reversal that would anticipate the need for additional staff, security, or other department resources to protect caregivers from a patient's violent outburst.

A vision screening device 104 is configured to perform an eye test to determine whether the subject 102 is suspected to have one or more ocular conditions and/or to provide an indication of one or more likelihoods for various types of behavior. In various implementations, the vision screening device 104 identifies the performance of the subject 102 on a vision test 108 output by an external medium 110. As used herein, the term “vision test,” and its equivalents, can refer to displayed information that can be used to assess the vision of an individual. For example, the vision test 108 may include a color deficiency test, which can also be referred to as a “color blindness” test. Examples of color deficiency tests include Ishihara tests and Color Vision Tests Made Easy (CVTME) tests. The vision test 108 may include one or more pictures that display a symbol (e.g., a number) in at least one first color and at least one second color as a background to the symbol. If the subject 102 has sufficient color sensitivity, the subject may see the symbol. If the subject 102 is color deficient, the subject 102 may be unable to discern the symbol.

In some cases, the vision test 108 includes a visual acuity test. According to some implementations, a visual acuity test displays symbols of with different sizes. The visual acuity of the subject 102 is determined based on the sizes at which the subject 102 can visually recognize one or more of the symbols. There are multiple types of visual acuity tests, such as near vision tests and distance vision tests. Near vision tests display the symbols at a relatively close distance from the eye of the subject 102, such as 35 centimeters (cm). The results of a near vision test are indicative of whether the subject 102 is nearsighted. Distance vision tests display the symbols at a relatively long distance from the eye of the subject 102, such as 6 meters (m). The results of a distance vision test are indicative of whether the subject is farsighted.

In various examples, the vision test 108 includes a reading speed test. For example, the vision test 108 displays multiple words. The speed at which the subject 102 reads the words corresponds to the reading speed of the subject 102. In some instances, the vision test 108 includes a reading comprehension test. The vision test 108 may display a passage of words. Upon reading the passage, the subject 102 may indicate what the passage discusses, thereby demonstrating whether the subject 102 adequately understands the passage. Reading speed tests and reading comprehension tests may be used to assess whether the subject 102 has a learning disability or other condition.

According to various implementations, the vision test 108 includes a concussion test. For instance, the vision test 108 may include a test described in U.S. Pat. No. 10,506,165, which is incorporated by reference herein in its entirety. For instance, the vision test 108 may include one or more symbols that the subject 102 focuses on visually. The vision screening device 104 may capture one or more images of the eyes of the subject 102 while the subject is focusing on the vision test 108. In some cases, the vision screening device 104 determines a pupil size of the subject 102 based on the image(s), and determines whether the subject 102 is predicted to have a concussion based on a pupil size of the subject 102.

In some cases, the vision test 108 is gamified for the subject 102. For example, the external medium 110 may display a shape (e.g., a butterfly) that moves along the external medium 110. The subject 102 may play a game by inputting feedback based on the position of the shape. For example, the subject 102 may “capture” a virtual butterfly displayed by the external medium 110 by controlling an input device (e.g., a touchscreen), and based on the feedback, the vision screening device 104 may evaluate the vision of the subject 102.

In various examples, the vision test 108 is displayed by the external medium 110. As used herein, the term “external medium,” and its equivalents, can refer to a device and/or object that is separate from a device used to identify the results of a vision test (e.g., the vision screening device 104). In some cases, the external medium 110 includes a substrate (e.g., a passive object), such as a projection screen reflecting a projection of the vision test 108, a poster displaying the vision test 108, a card displaying the vision test 108, or some other printed substrate displaying the vision test 108. As used herein, the term “substrate,” and its equivalents, can refer to a solid or semisolid material that can absorb and/or reflect light. In various examples, the external medium 110 includes an active device, such as a tablet computer or smartphone that displays the vision test 108 on a touchscreen, a smart TV that displays the vision test 108, a virtual reality (VR) headset that displays the vision test 108, an augmented reality device that displays the vision test 108, or some other computing device that displays the vision test 108 on a screen.

According to various implementations, the vision screening device 104 may be configured to assess the results of multiple different vision tests including the vision test 108 and/or the behavior prediction. To identify the vision test 108 among the multiple vision tests, the vision screening device 104 may identify a code 112 that is associated with the vision test 108. As used herein, the term “code,” and its equivalents, can refer to one or more symbols that indicate the identity of a vision test. In some examples, the code 112 is displayed on the external medium 110 with the vision test 108. For example, the vision screening device 104 includes a camera that captures an image of the code 112 on the external medium 110. As used herein, the term “image,” and its equivalents, can refer to a set of data including multiple pixels and/or voxels that respectively represent regions of a real-world scene. A two-dimensional (2D) image is represented by an array of pixels. A three-dimensional (3D) image is represented by an array of voxels. An individual pixel and/or voxel in an image is defined according to at least one value representing an amount and/or frequency of light emitted by the corresponding region in the real-world scene.

The external medium 110 may also include a prediction engine 124 that is used to perform the behavior likelihood scoring. Therefore, based at least in part on the analysis of the captured images from the vision screening device during the one or more vision tests, the vision screening device 104 (and/or a computing device associated with the device) may generate an output including a recommendation, prediction, evaluation, or other indication of the status of the patient and/or a prediction of certain behaviors such as outbursts, flight risk, violent behavior, etc. For example, the device may detect if a patient is impaired due to drug use and indicate a violence risk score based on the captured images. Notably, the vision screening device 104 may provide such indications earlier than a blood test may return a result indicative of such impairment or altered status and therefore the caregivers may be prepared to allocate resources as necessary before an incident occurs. The vision screening device 104 may be used as part of the routine physical assessment at triage on a patient when they enter an emergency department or other caregiving location.

The vision screening device 104 may automate an eye test typically performed by physicians and output an indication to caregivers with a more limited scope of practice. For instance, the output may include a risk score associated with a warning of violence propensity. In some examples, after the vision screening device is used to perform the test, the captured images or other such data gathered by the vision screening device can be used to determine one or more behavior scores indicative of likelihoods associated with particular behavior. The determination can be made on-board the vision screening device 104 and/or by a computing device communicably coupled (e.g., over a network) with the vision screening device and may include the use of a machine learning, artificial intelligence, or other algorithm to analyze the captured images. The algorithms or other such techniques may be trained using eye exam data tied to patient data that documents particular behaviors such as violent outbursts.

In some implementations, a signal indicative of the code 112 is transmitted from the external medium 110 to the vision screening device 104. For instance, the vision screening device 104 includes a transceiver that receives a signal (e.g., a wireless signal) indicative of the code 112 from the external medium 110.

The subject 102 may view the vision test 108 and produce feedback based on the vision test 108. As used herein, the term “feedback,” and its equivalents, can refer to data representing an individual's performance on a vision test. The feedback, for example, is detected by a feedback device 116. In some implementations, the feedback device 116 is part of the vision screening device 104 and/or the external medium 110. In various cases, the feedback device 116 includes a sensor configured to detect the feedback from the subject 102.

Various types of feedback can be detected by the feedback device 116. In some implementations, the feedback device 116 includes one or more touch sensors incorporated with the external medium 110. The feedback may be a touch of the subject 102 on at least a portion of the external medium 110. For example, the subject 102 may trace a symbol of the vision test 108, which is detected by the touch sensor(s) of the feedback device 116. In some cases, the subject 102 may touch an icon displayed on the external medium 110 that is detected by the feedback device 116 as the feedback from the vision test 108.

In various examples, the feedback device 116 includes one or more cameras that visually detect the feedback from the subject 102. For example, the vision test 108 may be a reading speed test and the camera(s) capture images of an eye of the subject 102 as the subject is reading the passage. The feedback may be the change in the gaze angle of the subject 102 over time. The camera(s) may capture images of the eyes to determine, using the prediction engine 124, one or more eye characteristics that may shift over time, and be used to determine eye and/or pupil characteristics that may be associated with particular propensities towards certain behavior, such as may be linked with an impaired mental state.

In some cases, the feedback device 116 includes other types of input devices that can detect feedback directly from the subject 102. For example, the feedback device 116 may include a microphone that detects the voice of the subject 102 that serves as the feedback about the vision test 108. In some examples, the feedback device 116 includes physical buttons, a keyboard, or any other device configured to detect an input signal indicative of the feedback from the subject 102.

According to some examples, the user 106 inputs the feedback from the subject 102 into the feedback device 116. For example, the subject 102 may audibly report the feedback to the user 106, who may manually input the feedback into the feedback device 116 using a button, keyboard, touch screen, or other input device.

The feedback device 116 may provide the feedback to the vision screening device 104. In various implementations, the vision screening device 104 may determine whether the subject 102 is suspected to have an ocular condition by analyzing the feedback in view of the vision test 108. In some cases, the entry in the test datastore 114 indicating the vision test 108 may further include a key associated with the vision test 108. For instance, if the vision test 108 is an Ishihara color deficiency test, the key may be the identity of the symbol that is displayed in the vision test 108. The feedback device 116 may compare the feedback to the key. In some implementations, the key is defined as a shape that is within a threshold distance (e.g., 1 centimeter) of the symbol. The feedback device 116 may determine whether the subject 102 traces a shape that is within the key. In some implementations, the subject 102 traces the symbol on a touchscreen, and the feedback may be highlighted on the touchscreen as the subject 102 is tracing the symbol. In some implementations, the subject 102 traces the symbol with a writing instrument (e.g., a marker, pen, or pencil) on a paper substrate. The highlighted and/or written feedback may be viewed manually. If the feedback matches the key, then the vision screening device 104 may determine that the subject 102 has passed the vision test 108. If the feedback is different than the key, then the vision screening device 104 may determine that the subject 102 has not passed the vision test 108. In various implementations, the vision screening device 104 may determine that the subject 102 is suspected to have an ocular condition based on determining that the subject 102 has not passed the vision test 108.

In some implementations, the key indicates a threshold that the vision screening device 104 compares to the feedback. For example, if the vision test 108 is a reading speed test, and the feedback represents a reading speed of the subject 102, the vision screening device 104 may compare the reading speed of the subject 102 to a threshold speed in order to determine whether the subject 102 is at an appropriate reading level or is suspected of having a learning disability.

The vision screening device 104 may perform additional tests on the subject 102 that are independent of the external medium. In various implementations, the vision screening device 104 performs an automated autorefraction assessment on the subject 102. For example, the vision screening device 104 may include at least one light source configured to project an infrared pattern on an eye of the subject 102. As used herein, the term “light source,” and its equivalents, can refer to an element configured to output light, such as a light emitting diode (LED) or a halogen bulb.

The vision screening device 104, in some instances, further includes at least one camera configured to capture an image of a reflection of the pattern from the eye of the subject 102. The vision screening device 104 may determine a condition of the subject 102 based on the reflection of the pattern. For example, the vision screening device 104 may determine that the subject has myopia, hyperopia, astigmatism, or a combination thereof, based on the reflection of the pattern. In some cases, the vision screening device 104 is or includes a specialized device, such as the Welch Allyn Spot Vision Screener by Hill-Rom Services, Inc. of Chicago, IL. In some cases, the vision screening device 104 performs a red reflex examination on the subject 102.

According to various implementations, the vision screening device 104 may output and/or store a result of the vision test 108 or the result of any other vision test identified by the vision screening device 104. The result, for example, is an indication of the feedback, a discrepancy between the feedback and the key, whether the subject 102 is suspected to have the ocular condition, or a combination thereof. In some implementations, the vision screening device 104 outputs the result to the user 106. For example, the vision screening device 104 may display the result on a screen and/or audibly output the result using a speaker. In some cases, the visions screening device 104 stores the result (e.g., with an indication of the identity of the subject 102).

In some cases, the vision screening device 104 determines an identity of the subject 102. For instance, the user 106 may input a code, name, or other identifier associated with the subject 102 into the vision screening device. The vision screening device 104 may generate and/or store the result with the identifier of the subject 102.

The vision screening device 104 may be communicatively coupled to an electronic medical record (EMR) system 118. In some cases, the vision screening device 104 transmits the result (and the identifier of the subject 102) to the EMR system 118. The EMR system 118 may include one or more servers storing EMRs of multiple individuals including the subject 102. As used herein, the terms “electronic medical record,” “EMR,” “electronic health record,” and their equivalents, can refer to a data indicating previous or current medical conditions, diagnostic tests, or treatments of a patient. The EMRs may also be accessible via computing devices operated by care providers. In some cases, data stored in the EMR of a subject is accessible to a user via an application operating on a computing device. For instance, the stored data may indicate demographics of a subject, parameters of the subject, vital signs of the subject, behavioral notes or indications of the subject (e.g., instances of outbursts, violence, or other behavior), notes from one or more medical appointments attended by the subject, medications prescribed or administered to the subject, therapies (e.g., surgeries, outpatient procedures, etc.) administered to the subject, results of diagnostic tests performed on the subject, subject identifying information (e.g., a name, birthdate, etc.), or any combination thereof. In various implementations, the EMR system 118 stores the feedback and/or result in an EMR associated with the subject 102.

In some examples, the vision screening device 104 transmits the result to one or more web server(s) 120. In various implementations, the web server(s) 120 may store indications of the results, including the behavior scores output by the prediction engine 124. In addition, the web server(s) 120 may output a website to an external computing device (not illustrated) indicating the result. In some cases, the external computing device may be operated by a parent of the subject 102, such that the parent may view the indication of the result by accessing the website. In some implementations, the web server(s) 120 further stores additional information about the vision test 108 and/or recommended follow-up care for the subject 102. For instance, based on the result, the website may indicate that the subject 102 should be seen by an optometrist and/or ophthalmologist for follow-up care.

Various elements of the system 100 communicate via one or more communication network(s) 122. The communication network(s) 122 include wired (e.g., electrical or optical) and/or wireless (e.g., radio access, BLUETOOTH, WI-FI, or near-field communication (NFC)) networks. The communication network(s) 122 may forward data in the form of data packets and/or segments between various endpoints, such as computing devices, medical devices, servers, and other networked devices in the system 100.

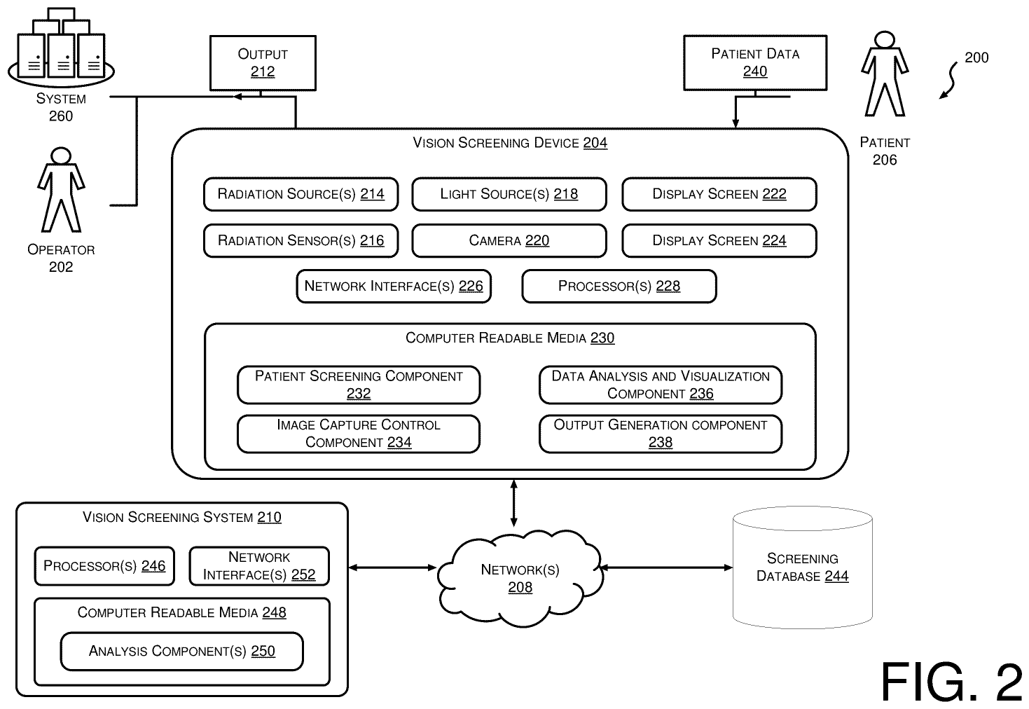

FIG. 2 illustrates a system 200 for administering vision screening tests, and in particular, screening tests for detection of diseases and/or abnormalities of eye(s), according to some implementations. As illustrated in FIG. 2, in some examples an operator 202 may administer vision screening tests, via a vision screening device 204, on a patient 206 to determine a behavior likelihood score and/or evaluate eye health of the patient 206. As described herein, the vision screening device 204 may perform one or more vision screening tests, including screening for particular impairments, altered mental states, screening for diseases and/or abnormalities of eye(s) when the eyes are illuminated by visible light. In addition, the vision screening device 204 may also be configured to perform other vision screening tests, such as a visual acuity test, a refractive error test, an accommodation test, dynamic eye tracking tests, color vision screening test and/or any other vision screening tests, configured to evaluate and/or diagnose the vision health of the patient 206. In examples, the vision screening device 204 may comprise a portable device configured to perform the one or more vision screening tests. Due to its portable nature, the vision screening device 204 may perform the vision screening tests at any location, from conventional screening environments, such as schools and medical clinics, to physician's offices, hospitals, eye care facilities, and/or other remote and/or mobile locations. It is also envisioned that the vision screening device 204 may be used for administering vision screening tests to all age groups, including newborns and young children and geriatric patients.

As described herein, the vision screening device 204 may be configured to perform one or more vision screening tests on the patient 206. In examples, one or more vision screening tests may include illuminating the eye(s) of the patient 206 with infrared or near-infrared (NIR) radiation, and capturing reflected radiation from the eye(s) of the patient 206. For example, U.S. Pat. No. 9,237,846, the entire disclosure of which is incorporated herein by reference, describes systems and methods for determining refractive error based on photorefraction using pupil images captured under different illumination patterns generated by near-infrared (NIR) radiation sources. In other examples, vision screening tests, such as the red reflex test, may include illuminating the eye(s) of the patient 206 with visible light, and capturing color image(s) of the eye(s) under visible light illumination. The vision screening device 204 may acquire data comprising color images and/or video data of the eye(s) under visible light illumination, and detect pupils, retinas, and/or lenses of the eye(s) of the patient 206. This data may be used to determine differences between left and right eyes, compare the captured images with standard images, or generate visualizations to assist the operator 202 or a clinician in diagnosing diseases and abnormalities of the eye(s) of the patient. The data may also be used to identify when a patient 206 may be under the influence of substances that may produce an altered mental state and may therefore result in particular patterns or predicted behavior. The vision screening device 204 may transmit the data, via a network 208, to a vision screening system 210 for analysis to determine an output 212 associated with the patient 206. Alternatively, or in addition, the vision screening device 204 may perform some or all of the analysis locally to determine the output 212. The output 212 may be provided to an operator 202 or other system 260, including, for example a security system, nursing station, management system, electronic medical record system, or other such system. Indeed, in any of the examples described herein, some or all of the disclosed methods may be performed in whole or in part by the vision screening device 204 independently (e.g., without the vision screening system 210 or its components), or by the vision screening system 210 independently (e.g., without the vision screening device 204 or its components). For instance, in some examples, the vision screening device 204 may be configured to perform any of the behavior likelihood determinations, vision screening tests, and/or other methods described herein without being connected to, or otherwise in communication with, the vision screening system 210 via the network 208. In other example, the vision screening system 210 may include one or more components that are similar to and/or the same as those included in the vision screening device 204, and thus, the vision screening system 210 may be configured to perform any of the behavior likelihood determinations, vision screening tests, and/or other methods described herein without being connected to, or otherwise in communication with, the vision screening device 204.

As shown schematically in FIG. 2, the vision screening device 204 may include one or more radiation source(s) 214 configured to perform functions associated with administering one or more vision screening tests. The radiation source(s) 214 may comprise individual radiation emitters, such as light-emitting diodes (LEDs), which may be arranged in a pattern to form an LED array. In examples, the radiation source(s) 214 may include near-infrared (NIR) radiation emitters, such as NIR LEDs, for measuring the refractive error of the eye(s) of the patient 206 using photorefraction methods. The NIR radiation emitters of the radiation source(s) 214 may also be used for measuring the gaze angle or gaze direction of the eye(s) of the patient 206. In addition, the radiation source(s) 214 may also include color LEDs for generating color stimuli for display to the patient 206 during a color vision screening test.

The vision screening device 204 may also include one or more radiation sensor(s) 216, such as infrared cameras, configured to capture reflected radiation from the eye(s) of the patient during the vision screening test(s). For example, the vision screening device 204 may emit, via the radiation source(s) 214, one or more beams of radiation, and may be configured to direct such beams at the eye(s) of the patient 206. The vision screening device 204 may then capture, via the radiation sensor(s) 216, corresponding radiation that is reflected back (e.g., from pupils of the eye(s)). In examples, the radiation sensor(s) 216 may comprise NIR radiation sensor(s) to capture reflected NIR radiation while the eye(s) of the patient 206 are illuminated by the NIR radiation source(s) 214. The data captured by the NIR radiation sensor(s) 216 may be used in the measurement of the refractive error and/or gaze angle(s) of the eye(s) of the patient 206. The data may include images and/or video of the pupils, retinas, and/or lenses of the eyes of the patient 206. In some examples, the images and/or video may be in grayscale (e.g., with values between 0 and 228, or between 0 and 256). The data may be captured intermittently, during specific periods of the vision screening test(s), or during the entire duration of the test(s). Additionally, the vision screening device 204 may process the image(s) and/or video data to determine change(s) in the refractive error and/or gaze angle(s) of the eye(s) of the patient 206. The grayscale images of the eye(s) captured under NIR illumination may also be used for screening for diseases and abnormalities of the eye(s) such as ametropia, strabismus, and occlusions. Such data may be used for determining characteristics of pupils, for instance including reactivity of pupils to illumination, dilation, motion, eye tracking, differences in tracking between the right and left eyes, and other such data that may be used to evaluate a likelihood of certain behavior based on the physical characteristics observed using the vision screening device.

In examples, the vision screening device 204 may further include visible white light source(s) 218 and camera 220 configured to capture color images and/or video of the eyes under illumination by the white light source(s) 218. The white light source(s) 218 may comprise light-emitting diodes (LEDs) such as an array of LEDs configured to produce white light e.g., a blue LED with a phosphor coating to convert blue light to white light, or a combination of red, blue, and green LEDs configured to produce white light by varying intensities of individual red, blue and green LED activation. Individual LEDs of the array of LEDs may be arranged in a pattern configured to be individually operable to provide illumination from different angles during the vision screening test(s). The white light source(s) 218 may also be configured to produce white light of different intensity levels. The camera 220 may be configured to capture white light reflected from the eyes of the patient to produce digital color images and/or video. The camera 220 may comprise a high-resolution, auto-focus digital camera with custom optics for imaging eyes in clinical applications, as described in further detail with reference to FIG. 2. The color images and/or video captured by the camera 220 may be stored in various formats, such as JPEG, BITMAP, TIFF, etc. (for images) and MP4, MOV, WMV, AVI etc. (for video). In some examples, pixel values in the color images and/or video may be in a RGB (red, green, blue) color space. The color images and/or video of the eye(s) captured under white light illumination may be used for screening for diseases and abnormalities of the eye(s) such as cataracts, media opacities in aqueous and vitreous humors, tumors, retinal cancers and detachment, and the like. In addition, the color images and/or video may be used in conjunction with the grayscale images captured under NIR illumination to generate visualizations to assist in the detection of a wide range of disease conditions of the eye(s). The color images may likewise be used for determining characteristics of pupils, for instance including reactivity of pupils to illumination, dilation, motion, eye tracking, differences in tracking between the right and left eyes, and other such data that may be used to evaluate a likelihood of certain behavior based on the physical characteristics observed using the vision screening device.

The vision screening device 204 may also include one or more display screen(s), such as display screen 222 and display screen 224, which may be color LCD (liquid crystal display), or OLED (organic light-emitting diode) display screens. The display screen 222 may be an operator display screen facing a direction towards the operator 202, configured to provide information related to the vision screening tests to the operator 202 such as the behavior likelihood score (e.g., outputting a score indicative of a likelihood for the patient 206 to exhibit particular behavior such as violence). In any of the examples described herein, the display screen 222 facing the operator 202 may be configured to display and/or otherwise provide the output 212 generated by the vision screening device 204 and/or generated by the vision screening system 210. The output 212 may include testing parameters, current status and progress of the screening test(s), measurements(s) determined during the test(s), image(s) captured or generated during the screening test(s), a diagnosis determined based on one or more tests, and/or a recommendation associated with the diagnosis. The display screen 222 facing the operator 202 may also display information related to or unique to the patient, and the patient's medical history.

In some examples, the vision screening device 204 may also include a display screen 224 facing in a direction towards the patient 206, and configured to display content to the patient 206. The content may include attention-attracting images and/or video to attract attention of the patient and hold the patient's gaze towards the vision screening device 204. Content corresponding to various vision screening test(s) may also be presented to the patient 206 on the display screen 224. For example, the display screen 224 may display color stimuli to the patient 206 during a color vision screening test, or a Snellen eye chart during a visual acuity screening test. The display screens 222, 224 may be integrated with the vision screening device 204, or may be external to the device, and under computer program control of the vision screening device 204. The display screen 224 may be used to gather information about the state of the patient and keep the patient 206 focused while the vision screening is performed.

The vision screening device 204 may transmit the data captured by the radiation sensor(s) 216 and the camera 220, via the network 208, using network interface(s) 226 of the vision screening device 204. In addition, the vision screening device 204 may also similarly transmit other testing data associated with the vision screening test(s) being administered, (e.g., type of test, duration of test, patient identification and the like). The network interface(s) 226 of the vision screening device 204 may be operably connected to one or more processor(s) 228 of the vision screening device 204, and may enable wired and/or wireless communications between the vision screening device 204 and one or more components of the vision screening system 210, as well as with one or more other remote systems and/or other networked devices. For instance, the network interface(s) 226 may include a personal area network component to enable communications over one or more short-range wireless communication channels, and/or a wide area network component to enable communication over a wide area network. In any of the examples described herein, the network interface(s) 226 may enable communication between, for example, the processor(s) 228 of the vision screening device 204, and the vision screening system 210, via the network 208. The network 208 shown in FIG. 2 may be any type of wireless network or other communication network known in the art. Examples of network 208 include the Internet, an intranet, a wide area network (WAN), a local area network (LAN), and a virtual private network (VPN), cellular network connections and connections made using protocols such as 802.11a, b, g, n and/or ac.

The vision screening system 210 may be configured to receive data, from the vision screening device 204 and via the network 208, collected during the administration of the vision screening test(s). In some examples, based at least in part on processing the data, the vision screening system 210 may determine the output 212 associated with the patient 206. For example, the output 212 may include a score indicative of particular behavior such as violence, flight risk, withdrawal symptoms, theft, or other behaviors. The output 212 may also include a recommendation and/or diagnosis associated with eye health of the patient 206, based on an analysis of the color image data and/or NIR image data indicative of diseases and/or abnormalities associated with the eye(s) of the patient 206. The vision screening system 210 may communicate the output 212 to the processor(s) 228 of the vision screening device 204 via the network 208. As noted above, in any of the examples described herein one or more such recommendations, diagnoses, or other outputs may be generated, alternatively or additionally, by the vision screening device 204. The vision screening system 210 may also communicate the output 212 to the system 260.

As described herein, a processor, such as the processor(s) 228, can be a single processing unit or a number of processing units, and can include single or multiple computing units or multiple processing cores. The processor(s) 228 can be implemented as one or more microprocessors, microcomputers, microcontrollers, digital signal processors, central processing units, state machines, logic circuitries, and/or any devices that manipulate signals based on operational instructions. For example, the processor(s) 228 can be one or more hardware processors and/or logic circuits of any suitable type specifically programmed or configured to execute the algorithms and processes described herein. As shown schematically in FIG. 2, the vision screening device 204 may also include computer-readable media 230 operably connected to the processor(s) 228. The processor(s) 228 can be configured to fetch and execute computer-readable instructions stored in the computer-readable media 230, which can program the processor(s) 228 to perform the functions described herein.

The computer-readable media 230 may include volatile and nonvolatile memory and/or removable and non-removable media implemented in any type of technology for storage of information, such as computer-readable instructions, data structures, program modules, or other data. Such computer-readable media 230 can include, but is not limited to, RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. The computer-readable media 230 can be a type of computer-readable storage media and/or can be a tangible non-transitory media to the extent that when mentioned, non-transitory computer-readable media exclude media such as energy, carrier signals, electromagnetic waves, and signals per se.

The computer-readable media 230 can be used to store any number of functional components that are executable by the processor(s) 228. In examples, these functional components comprise instructions or programs that are executable by the processor(s) 228 and that, when executed, specifically configure the one or more processor(s) 228 to perform actions associated with one or more of the vision screening tests used for the detection and diagnosis of diseases and abnormalities of the eye(s). For example, the computer-readable media 230 may store one or more functional components for administering vision screening tests, such as a patient screening component 232, an image capture control component 234, a data analysis and visualization component 236, and/or an output generation component 238, as illustrated in FIG. 2. At least some of the functional components of the vision screening device 204 will be described in detail below.

In examples, the patient screening component 232 may be configured to store and/or access patient data 240 associated with the patient 206. For example, the patient data 240 may include demographic information such as name, age, ethnicity, and the like. When the vision screening device 204 and/or vision screening system 210 initiates a vision screening test, the patient 206 may provide, or the operator 202 may request, from the patient 206 or a guardian of the patient 206 the patient data 240 regarding the patient's demographic information, medical information, preferences, and the like. In such examples, the operator 202 may request the data while the screening is in progress, or before the screening has begun. In some examples, the operator 202 may be provided with predetermined categories associated with the patient 206, such as predetermined age ranges (e.g., newborn to six months, six to twelve months, one to five years old, etc.), and may request the patient data 240 in order to select the appropriate category associated with the patient 206. In other examples, the operator 202 may be provided a free form input associated with the patient data 240. In still further examples, an input element may be provided to the patient 206 directly.

Alternatively, or in addition, the vision screening device 204 and/or vision screening system 210 may determine and/or detect the patient data 240 during the vision screening test. For example, the vision screening device 204 may include one or more digital cameras, motion sensors, proximity sensors, or other image capture devices configured to collect images and/or video data of the patient 206, and one or more processors of the vision screening device 204 may analyze the data to determine the patient data 240, such as the age category of the patient 206 or a distance of the patient 206 from the screening device. For example, the vision screening device 204 may be equipped with a range finder, such as an ultra-sonic range finder, an infrared range finder, and/or any other proximity sensor that may be able to determine the distance of the patient 206 from the screening device.

Alternatively, or in addition, the vision screening device 204 may be configured to transmit the images/video data to the vision screening system 210, via the network 208, for analysis to determine the patient data 240. Further, the patient screening component 232 may be configured to receive, access, and/or store the patient data 240 associated with the patient 206 and/or additional patients. For example, the patient screening component 232 may store previous patient information associated with the patient 206 and/or other patients. For instance, the patient screening component 232 may store previous screening history of the patient 206, including data from previous screening such as color images, NIR images, and/or video of the eye(s) of the patient 206. The patient screening component 232 may receive the patient data 240 and/or may access such information via the network 208. For example, the patient screening component 232 may access an external database, such as screening database 244, storing data associated with the patient 206 and/or other patients. The screening database 24 may be configured to store the patient data 240 in association with a patient ID. When the operator 202 and/or the patient 206 enters the patient ID, the patient screening component 232 may access or receive the patient data 240 stored in association with the patient ID of the patient 206.

In examples, the patient screening component 232 may be configured to determine the vision screening test(s) to administer to the patient 206 based at least in part on the patient data 240. For example, the patient screening component 232 may utilize the patient data 240 to determine a testing category that the patient 206 belongs to (e.g., a testing category based on age, medical history, etc.). The patient screening component 232 may determine the vision screening test(s) to administer based on the testing category. For example, if the patient data 240 indicates that the patient is a newborn, the selected vision screening test(s) may include screening for congenital conditions of the eye such as congenital cataracts, retinoblastoma, opacities of the cornea, strabismus and the like. In addition, eye abnormalities may be associated with systemic inherited diseases such as Marfan syndrome and Tay-Sachs disease. For example, a screening test for a characteristic red spot in the eye may indicate Tay-Sachs disease. As another example, if the patient data 240 indicates that the patient is above fifty years old, the patient screening component 232 may determine that the vision screening test(s) include screening for onset of cataracts, macular degeneration and other age-related eye diseases.

The patient screening component 232 may also determine vision screening test(s) based on the patient's medical history. For example, the screening database 244 may store, in the patient data 240, medical history associated with previous vision screening tests of the patient 206, including test results, images of the eye(s), measurements, recommendations, and the like. The patient screening component 232 may access the patient data 240 including medical history from the screening database 244 and determine vision screening test(s) to administer to monitor status and changes in previously detected vision health issues. For example, if a progressive eye disease, such as onset of cataracts or macular degeneration, was detected in a previous screening, further screening may be administered to track the development of the disease. As another example, if the patient 206 had surgery for removal of a tumor of the eye(s), the vision screening test(s) may include screening for further tumors or scarring in the eye(s). The patient screening component 232 may determine a list of vision screening tests to be administered to the patient 206 during a vision screening session, and keep track of the vision screening tests that have already been administered during the vision screening session, as well remaining vision screening tests on the list of vision screening tests to be administered.

The patient screening component 232 may also determine the behavior likelihood score indicative of if a patient is impaired and may be used to screen or identify patients that may be at a higher risk for potentially violent behavior or other predicted behavior, based on the captured images.

In some examples, the computer-readable media 230 may additionally store an image capture control component 234. The image capture control component 234 may be configured to operate the radiation source(s) 214, the radiation sensor(s) 216, the white light source(s) 218, and the camera 220 of the vision screening device 204, so that images of the eye(s) are captured under specific illumination conditions required for each particular vision screening test(s). As discussed, the radiation source(s) 214 may include NIR LEDs for illuminating the eye(s) during capture of grayscale images for measuring the refractive error and/or gaze angle of the eye(s) of the patient 206, and the white light source(s) 218 may include white light LEDs for illuminating the eye(s) during capture of color images of the eye(s) by the camera 220. In examples, the image capture control component 234 may generate commands to operate and control the individual radiation sources, such as LEDs of the NIR LEDs, as well as the LEDs of the white light source 218. Control parameters of the LEDs may include intensity, duration, pattern and cycle time. For example, the commands may selectively activate and deactivate the individual LEDs of the radiation sources 214 and white light sources 218 to produce illumination from different angles as needed by the vision screening test(s) indicated by the patient screening component 232. The image capture control component 234 may activate the NIR LEDs of the radiation source(s) 214 used for measuring the refractive error and/or gaze angle of the eye(s) of the patient 206 in synchronization with the capture of images of the eye(s) by the radiation sensors(s) 216 during the performance of a vision screening test. Similarly, the image capture control component 234 may activate the LEDs of the white light source(s) 218 in synchronization with the capture of color images of the eye(s) by the camera 220.

The individual radiation sources, such as LEDs, of the radiation source(s) 214 or the white light source(s) 218 may be controlled by the image capture control component 234 according to control parameters stored in the computer-readable media 230. For instance, control parameters may include intensity, duration, pattern, cycle time, and so forth, of the NIR LEDs of the radiation source(s) 214 and/or the LEDs producing white light of the white light source(s) 218. For example, the image capture control component 234 may use the control parameters to determine a duration that individual LEDs of the radiation source(s) 214, 218 emit radiation (e.g., 50 milliseconds, 200 milliseconds, 200 milliseconds, etc.). Additionally, the image capture control component 234 may utilize the control parameters to alter an intensity and display pattern of NIR LEDs of the radiation source(s) 214 for the determination of refractive error of the eye(s) based on photorefraction and/or gaze angle of the eye(s). With respect to intensity, the image capture control component 234 may control parameters to direct the LEDs of the white light source(s) 218 to emit light at an intensity that is bright enough to capture a color image of the eye(s) using the camera 220, while also limiting brightness to avoid or reduce pupil constriction or accommodation. The image capture control component 234 may also control the intensity of the white light source(s) 218 to gradually increase the intensity at a certain rate while activating the camera 220 to capture images and/or video of the eyes to record response of the pupils of the patient's eyes to the increasing intensity of illumination.

Further, the image capture control component 234 may order the emission of radiation from the source(s) 214, 218 so that the NIR LEDs are activated and the images of the eye(s) under NIR radiation are captured before the activation of the LEDs of the white light source(s) 218. In some examples, this ordering may prevent the constriction of the pupils of the eye(s) in response to white light impinging upon them, and/or may allow for the capture of images of the internal structures of the eye(s) without the need for dilating the pupils of the patient 206. In some examples, the image capture control component 234 may additionally control the radiation source(s) to generate patterns such as circular patterns, alternating light patterns, flashing patterns, patterns of shapes such as circles or rectangles, and the like to attract the attention of the patient 206, and/or control color LEDs of the radiation source(s) 214, 218 to display color stimuli such as color dot patterns to the patient 206 during vision screening.

The image capture control component 234 may also control the radiation sensor(s) 216 and the camera 220 to capture images and/or video of the eye(s) of the patient 206 during the administration of the vision screening test(s). For example, the radiation sensor(s) 216 may capture data indicative of reflected radiation from the eye(s) of the patient 206 during the activation of one or more of the radiation source(s) 214. The data may include grayscale image data and/or video data of the eye(s). The image capture control component 234 may synchronize the camera 220 to capture color image(s) and/or video data of the eye(s) with the activation of the white light source(s) 218 so that the eye(s) are illuminated by white light radiation during the capture of the color image and/or video data. In some examples, images of the left and the right eye may be captured under different illumination conditions (e.g., from a different individual source), so that the relative angle of illumination with the optical axis of the particular eye is the same for the left and the right eye. In other examples, images of both eyes may be captured simultaneously under the same illumination. As described herein, the image capture control component 234 of the vision screening device 204 may generate grayscale images of the eye(s) illuminated under NIR radiation, and color images of the eye(s) illuminated under white light. Capturing both the grayscale images and the color images may enable the detection of a wider range of diseases and abnormalities of the eyes.

In some examples, the computer-readable media 230 may also store a data analysis and visualization component 236. The data analysis and visualization component 236 may be configured to analyze the image and/or video data collected, detected, and/or otherwise captured by components of the vision screening device 204 (e.g., by the radiation sensor(s) 216, and the camera 220) during one or more vision screening tests. For example, the data analysis and visualization component 236 may analyze the data to determine location of the pupils of the eye(s) in the images, and identify a portion of the image(s) corresponding to the pupil (e.g., pupil image(s)). The data analysis and visualization component 236 may analyze the pupil image(s) to determine characterizations of appearance of the pupil(s) in the pupil image(s). For example, in the instance of the color image(s) captured by the camera 220, the characterizations may include values corresponding to an average color, variance of color, measure of uniformity, presence of inclusions, and the like. In the instance infrared image(s) captured by the radiation sensor(s) 216, the characterizations may include average grayscale value and variance of grayscale values, instead of the color, in addition to measures of uniformity and the presence of inclusions. The data analysis and visualization component 236 may further compare the left pupil image(s) and the right pupil image(s) to determine differences in appearance between the left and right pupils. For example, the differences may correspond to a difference in average color value, average grayscale value, or uniformity between the left pupil image(s) and right pupil image(s). In normal eyes, an expected value of a characteristic (e.g., average color value, grayscale value, measure of uniformity, etc.) associated with one pupil image may be approximately same as a value of the characteristic in the other pupil image. The data analysis and visualization component 236 may also compare the pupil image(s) with standard pupil image(s) and/or pupil image(s) of the patient 206 captured during previous vision screening(s) to determine differences in appearance, such as differences in average color value or grayscale value, differences in the measure of uniformity, differences in detected inclusions, and the like. In such examples, an expected value of a characteristic of the pupil image(s) may correspond to the value of the characteristic in the standard pupil image(s) or previously-captured pupil image(s) of the patient 206. In any of the examples above, all captured image(s) or a subset of the captured grayscale and/or color images may be used to determine differences. In some examples, grayscale image(s) may not be used, and the difference may be determined based on the color image(s). It is to be noted that pixels of grayscale images may also be considered to have a color value, wherein the color value is determined by using the same grayscale value for each of the three color channels (e.g., RGB). For example, a pixel with a grayscale value of 228, may be determined to have a color value of (128, 228, 228) in the RGB color space. The data analysis and visualization component 236 may also apply additional image processing steps to the grayscale image(s) and/or the color image(s) which may improve detection of disease states. For example, images may be sharpened, specific colors may be boosted or attenuated, color or brightness of the images may be balanced, and the like.

Further, the data analysis and visualization component 236 may be configured to receive, access, and/or analyze standard data associated with vision screening. For example, the data analysis and visualization component 236 may be configured to access or receive data from one or more additional databases (e.g., the screening database 244, a third-party database, etc.) storing testing data, measurements, and/or values indicating various thresholds or ranges within which measured values should lie. The data from the additional databases may also be used to identify ranges of different characteristics that may be associated with an impaired mental state, use of substances, or other such indications that may be used in performing the behavior likelihood determination. Such thresholds or ranges may be associated with patients having normal vision health, and may be learned or otherwise determined from standard testing. The data analysis component and visualization component 236 may utilize the standard data for comparison with the average values and differences determined during the vision screening test(s) as described above. For example, the standard data may indicate a threshold or a range for a difference between color values of the left and right pupil images, where a difference greater than the threshold, or outside the range, corresponds to an abnormality in the eye(s) of the patient. Alternatively or in addition, the data analysis and visualization component 236 may access a previous vision screening of the patient 206 and compare the values and differences with corresponding data from the previous screening(s). For example, an average color value of the pupil may be compared with an average color value from a previous screening to determine a difference. This difference may then be compared with standard thresholds or ranges to determine presence of an abnormality or changes in the physical conditions of the patient 206. Separate threshold(s) and/or range(s) may be indicated in the standard data for different types of diseases and abnormalities. In addition, the threshold(s) and/or range(s) associated with the vision screening test may also be based on the testing category of the patient 206 (e.g., the age group or medical history of the patient 206), where the threshold(s) and/or range(s) may be different for different testing categories. The data analysis and visualization component 236 may store as a part of the patient data 240, images and/or video captured or generated during the vision screening test(s), measurements associated with the vision screening test(s), test results, and other data in a database (e.g., in the screening database 244) for comparison of data over time to monitor vision health status and changes in vision health. In some examples, the stored images may include images of the face or partial face (e.g., eyes and part of nose) of the patient 206.

Based on the comparison with a threshold and/or range described above, the data analysis and visualization component 236 may generate a behavior likelihood score for the patient 206 in addition to vision test results. For example, a score with respect to behavior associated with violence may be displayed as a numerical value on a scale of zero to one hundred, or may be provided as a rating of the prediction for violence being low, medium, high, or extreme, with thresholds for the behavior likelihood score associated with each of the ranges.

In examples, the data analysis and visualization component 236 may utilize one or more machine learning techniques to generate a behavior likelihood score in addition to diagnosis of specific diseases and/or types of abnormalities. For example, machine learning (ML) models may be trained with normal images of eyes, and images of eyes labeled with data indicative of behavior exhibited by the individual (e.g., as stored in patient medical record data) as well as labels exhibiting various disease conditions and abnormalities. The trained ML model(s) may then generate an output indicating the behavior likelihood score as well as a disease or abnormality diagnosis when provided, as input, an image of the eye captured during the vision screening of the patient 206. In such examples, the data analysis and visualization component 236 may directly generate the output by providing an image of the eye as input to the trained ML model(s), without computing differences between pupil images or applying comparisons with a threshold and/or range. In some examples, the images may be used to determine one or more characteristics, such as pupil characteristics over the duration of the test that may then be provided, either with or in place of the image data, to the trained ML model(s). In some examples, a plurality of trained ML model(s) may be used, each ML model being trained to determine behavior likelihood scores for various behavioral profiles, as may be determined by a caregiver facility. The data analysis and visualization component 236 may provide an image of the eye as input to each ML model of the plurality of trained ML model(s) for determining the behavior likelihood scores. In examples, the ML models may be neural networks, including convolutional neural networks (CNNs). In other examples, the ML models can also include regression algorithms, decision tree algorithms, Bayesian classification algorithms, clustering algorithms, support vector machines (SVMs) and the like.

The computer-readable media 230 may additionally store an output generation component 238. The output generation component 238 may be configured to receive, access, and/or analyze data from the data analysis and visualization component 236, and generate the output 212. For example, the output generation component 238 may utilize the behavior likelihood scores of the data analysis and visualization component 236 to generate a recommendation in the output 212. The recommendation may indicate a course of action for treating the patient 206, for allocating resources at the facility, for example to prepare to handle a particular behavior that is predicted to be forthcoming based on the behavior likelihood scores, and other such outputs. The output 212 may be presented to the operator of the device via an interface of the device (e.g., on the display screen 222 of the vision screening device 204). In examples, the operator display screen may not visible to the patient, e.g., the operator display screen may be facing in a direction opposite the patient. The output generation component 238 may also store the output 212, which may include a recommendation, diagnosis, measurements, captured images/video and/or the generated visualizations in a database, such as the screening database 244, for evaluation by a clinician, or for access during subsequent screening(s) of the patient 206. The screening database 244 may provide access to authorized medical professionals to enable printing of reports or further assessment of the data related to the screening of the patient 206.

Although FIG. 2 illustrates processor(s) 228 and computer-readable media 230 storing a patient screening component 232, an image capture control component 234, a data analysis and visualization component 236, an output generation component 238 and/or other components and/or other items as components of the vision screening device 204, in any of the examples described herein, the vision screening system 210 may include similar components and/or the same components. In such examples, the vision screening system 210 may include processor(s) 246 and computer-readable memory 248 that are configured to perform the functions of some or all of the components in the computer-readable memory 230 of the vision screening device 204. For example, one or more of the components of the computer-readable memory 230 may be included in analysis component(s) 250 of computer-readable memory 248 and be executable by the processor(s) 246. In such examples, the vision screening system 210 may communicate with the vision screening device 204 using network interface(s) 252, and via the network 208, to receive data from the vision screening device 204 and send results (e.g., output 212), back to the vision screening device 204. The vision screening system 210 may be implemented on a computer proximate the vision screening device 204, or may be at a remote location. For example, the vision screening system 210 may be implemented as a cloud service on a remote cloud server.

The network interface(s) 252 may enable wired and/or wireless communications between the components and/or devices shown in system 200 and/or with one or more other remote systems, as well as other networked devices. For instance, at least some of the network interface(s) 252 may include a personal area network component to enable communications over one or more short-range wireless communication channels. Furthermore, at least some of the network interface(s) 252 may include a wide area network component to enable communication over a wide area network. Such network interface(s) 252 may enable, for example, communication between the vision screening system 210 and the vision screening device 204 and/or other components of the system 200, via the network 208. For instance, the network interface(s) 252 may be configured to connect to external databases (e.g., the screening database 244) to receive, access, and/or send screening data using wireless connections. Wireless connections can include cellular network connections and connections made using protocols such as 802.11a, b, g, and/or ac. In other examples, a wireless connection can be accomplished directly between the vision screening device 204 and an external system using one or more wireless protocols, such as Bluetooth, Wi-Fi Direct, radio-frequency identification (RFID), infrared signals, and/or Zigbee. Other configurations are possible. The communication of data to an external database can enable report printing or further assessment of the patient's visual test data. For example, data collected and corresponding test results may be wirelessly transmitted and stored in a remote database accessible by authorized medical professionals.

It should be understood that, while FIG. 2 depicts the system 200 as including a single vision screening system 210, in additional examples, the system 200 may include any number of local or remote vision screening systems substantially similar to the vision screening system 210, and configured to operate independently and/or in combination, and configured to communicate via the network 208.

As discussed herein, FIG. 2 depicts a vision screening device 204 that includes components for administering vision screening tests to a patient. In some examples, one or more components may be implemented on a remote vision screening system 210 communicating with the vision screening device 204 over a network 208. The vision screening device 204 and its components are described in detail with reference to the remaining figures.

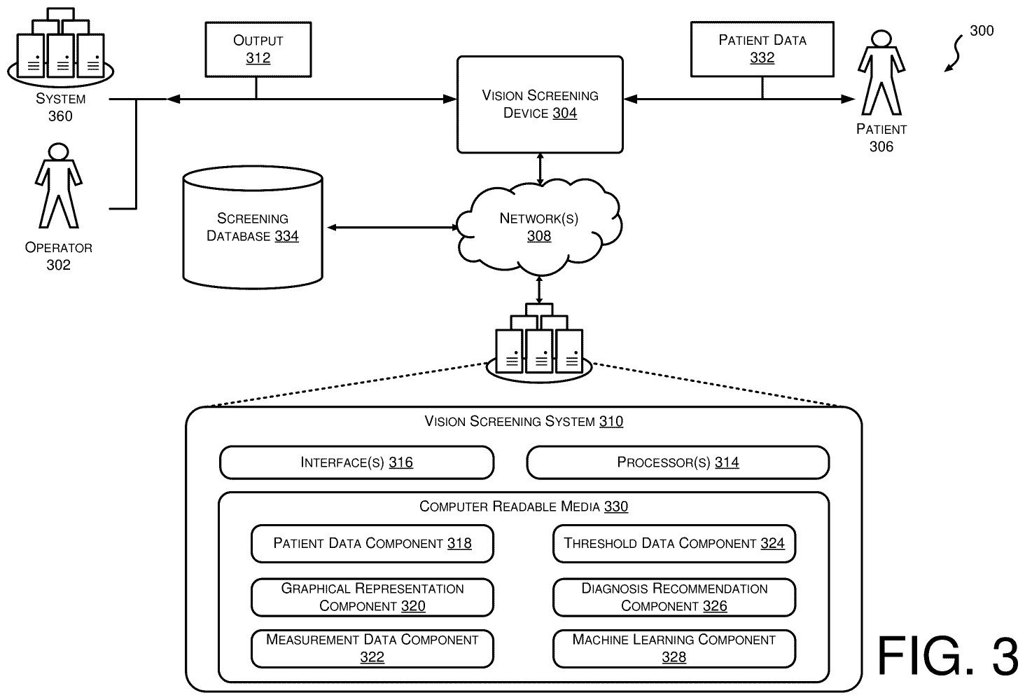

FIG. 3 illustrates an example environment for vision screening according to some implementations. As illustrated in FIG. 3, the environment 300 includes an operator 302 administering a vision screening, via a vision screening device 304, on a patient 306 to determine vision health of the patient 306. As described herein, the vision screening device 304 may perform one or more vision screening tests to determine one or more measurements associated with the patient 306 and provide the measurement(s), via a network 308, to a vision screening system 310 for analysis. In response, the vision screening system 310 may analyze the measurement(s) to diagnosis the vision health of the patient 306. Additionally, as described herein, the vision screening system may analyze the sensor data and/or measurement(s) to provide one or more behavior likelihood score indications indicative of probabilities for particular behavior from the patient 306. It should be understood that, while FIG. 3 depicts one system, vision screening system 310, the environment 300 may include any number of systems configured to operate independently and/or in combination and configured to communicate with each other via the network 308. The components of the vision screening system 310 may be described in detail below.

In examples, the vision screening system 310 may include one or more processor(s) 314, one or more network interface(s) 316, and computer-readable media 330. The computer-readable media 330 may store one or more functional components that are executable by processor(s) 314 such as a patient data component 318, a graphical representation component 320, a measurement data component 322, a threshold data component 324, a diagnosis recommendation component 326, and/or a machine learning component 328. At least some of the components, modules, or instructions of the computer-readable media 330 may be described below.

In examples, the vision screening device 304 may include a stationary or portable device configured to perform one or more vision screening tests on the patient 306. For example, the vision screening device 304 may be configured to perform a visual acuity test, a refractive error test, an accommodation test, dynamic eye tracking tests, and/or any other vision screening tests configured to evaluate and/or diagnose the vision health of the patient 306. Due to its stationary or portable nature, the vision screening device 304 may perform the vision screening tests at any location, from conventional screening environments, such as schools and medical clinics, to remote and/or mobile locations.

As described herein, the vision screening device 304 and/or vision screening system 310 may be configured to perform accommodation and refractive error testing on the patient 306. For example, refractive error and accommodation testing may include displaying a visual stimulus, such as a light or graphical representation, configured to induce a strain to the eyes of the patient 306. In response, the vision screening device 304 may detect the pupils and/or lenses of the eyes of the patient 306, acquire images and/or video data of the pupils/lenses, and the like, and may transmit the vision screening data, via the network 308, to the vision screening system 310 for analysis. Alternatively, or in addition, the vision screening device 304 may perform the analysis locally.

In examples, the vision screening device 304 may be configured to perform visual acuity testing and/or dynamic eye tracking tests. For example, the vision screening device 304 and/or the vision screening system 310 may be configured to perform visual acuity testing, which includes determining an optotype, determining a distance of the patient 306 from the vision screening device 304, and/or displaying a dynamic optotype to the patient 306. The dynamic eye tracking test may include generating a graphical representation, such as a graphic scene or text, for display to the patient 306 and monitoring the movement of the eye, acquire images and/or video data of the eyes, and the like, and may transmit the vision screening data, via the network 308, to the vision screening system 310 for analysis. Alternatively, or in addition, in some examples, the vision screening device 304 may analyze the vision screening data locally.

In examples, the patient data component 318 may be configured to store and/or access data associated with the patient 306. For example, the patient 306 may provide data, such as patient data 332, upon initiating a vision screening test. For instance, when the vision screening device 304 and/or vision screening system 310 initiates a vision screening test, the patient 306 may provide, or the operator 302 may request, the patient data 332 regarding the patient's demographic information, physical characteristics, preferences, and the like. For example, the patient 306 may provide demographic information such as name, age, ethnicity, and the like. The patient 306 may also provide physical characteristic information such as height of the patient 306. In such examples, the operator 302 may request the data while the screening is in progress, or before the screening has begun. In some examples, the operator 302 may be provided with predetermined categories associated with the patient 306, such as predetermined age ranges (e.g., six to twelve months, one to five years old, etc.), and may request the patient data 332 in order to select the appropriate category associated with the patient 306. In other examples, the operator 302 may provide a free form input associated with the patient data 332. In still further examples, an input element may be provided to the patient 306 directly.

Alternatively, or in addition, the vision screening device 304 and/or vision screening system 310 may determine and/or detect the patient data 332 during the vision screening test. For example, the vision screening device 304 may be configured to generate image and/or video data associated with the patient 306 at the onset of the vision screening test. For example, the vision screening device 304 may include one or more digital cameras, motion sensors, proximity sensors, or other image capture devices configured to collect images and/or video data of the patient 306, and one or more processors of the vision screening device 304 may analyze the data to determine the patient data 332, such as the height of the patient 306 or the distance of the patient 306 from the screening device. For example, the vision screening device 304 may be equipped with a range finder, such as an ultra-sonic range finder, an infrared range finder, and/or any other proximity sensor that may be able to determine the distance of the patient 306 from the screening device.

Alternatively, or in addition, the vision screening device 304 may be configured to transmit the images/video data to the vision screening system 310, via the network 308, for analysis to determine the patient data 332. For example, the vision screening device 304 may transmit the image/video data to the vision screening system 310 and the patient data component 318 may be configured to analyze the data to determine the patient data 332. Still further, the patient data component 318 may be configured to receive, access, and/or store patient data 332 associated with the patient 306 and/or additional patients. For example, the patient data component 318 may store previous patient information associated with the patient 306 and/or other patients who have utilized the vision screening system 310. For instance, the patient data component 318 may store previous patient preferences, screening history, and the like. The patient data component 318 may receive the patient data 332 and/or may access such information via the network 308. For example, the patient data component 318 may access an external database, such as screening database 334, storing data associated with the patient 306 and/or other patients. For example, the screening database 334 may be configured to store patient data 332 stored in association with a patient ID. When the operator 302 and/or patient 306 enters the patient ID, the patient data component 318 may access or receive the patient data 332 stored in association with the patient ID and the patient 306.

In addition, the computer-readable media 330 may store a graphical representation component 320. The graphical representation component 320 may be configured to generate or determine a graphical representation for display to the patient 306, via the vision screening device 304, during the vision screening process. For example, the graphical representation component 320 may be configured to receive and/or access patient data 332 from the patient data component 318 to determine a graphical representation to generate and/or display to the patient 306. As an example, the graphical representation component 320 may utilize the patient data 332 to determine a testing category that the patient 306 belongs to (e.g., a testing category based on age, height, etc.). Based on the patient data 332, the testing category, and/or the vision screening to be performed, the graphical representation component 320 may determine an existing graphical representation or generate a graphical representation for display to the patient 306.

The computer-readable media 330 may additionally store a measurement data component 322. The measurement data component 322 may be configured to receive, access, and/or analyze testing data collected and/or detected by the vision screening device 304 during the vision screening. For example, the measurement data component 322 may be configured to receive, via the network 308, video data generated by the vision screening device 304 of the patient 306 during the vision screening and while the graphical representation is being displayed. The measurement data component 322 may analyze the video data to determine one or more measurements associated with the patient 306, such as the gaze of the patient throughout the screening, a location of the patient's pupils at points in time of viewing the graphical representation, a diameter of the pupils, an accommodation of the lens, motion information associated with the eyes of the patient 306, and the like. Alternatively, or in addition, the measurement data component 322 may be configured to receive and/or access measurement data that has been determined by the vision screening device 304 locally.

Further, the computer-readable media 330 may be configured to store a threshold data component 324. The threshold data component 324 may be configured to receive, access, and/or analyze threshold data associated with standard testing results. For example, the threshold data component 324 may be configured to access, or receive data from, a third-party database storing testing data and/or measurements, or a range of values indicating a threshold within which testing values should lie, associated with patients having normal vision health with similar testing conditions. For example, for each testing category, standard testing data may be accessed or received by the threshold data component 324 and may be utilized for comparison against the measurement data stored by the measurement data component 322. For instance, the threshold data associated with the toddler testing category may include standard pupil measurements, and/or a threshold range of values which the testing values should not exceed or fall below (e.g., a standard value range) for toddlers when displayed each graphical representation. For example, when testing for accommodation in the patient 306, the threshold data component 324 may be configured to store information associated with the amplitude of accommodation and age (e.g., Donder's Table).

Alternatively, or in addition, the threshold data component 324 may be configured to utilize one or more machine learning techniques to determine threshold data associated with each testing category and/or graphical representation. For example, the threshold data component 324 may access and/or receive historical vision screening data from the screening database 334 and may utilize this data to train one or more machine learning models to determine standard testing measurements for each testing category. For example, machine learning component(s) (not shown) of the threshold data component 324 may execute one or more algorithms (e.g., decision trees, artificial neural networks, association rule learning, or any other machine learning algorithm) to train the system to determine the one or more threshold values based on historical vision screening data. In examples, the machine learning component(s) may execute any type of supervised learning algorithms (e.g., nearest neighbor, Naïve Bayes, Neural Networks, unsupervised learning algorithms, semi-supervised learning algorithms, reinforcement learning algorithms, and so forth).

The computer-readable media 330 may additionally store a diagnosis recommendation component 326. The diagnosis recommendation component 326 may be configured to receive, access, and/or analyze measurement data from the measurement data component 322 and/or threshold data from the threshold data component 324 for comparison. For example, the diagnosis recommendation component 326 may utilize the threshold data, learned or otherwise determined, for comparison against the measurement data to determine if the patient 306 is exhibiting normal vision behavior. For example, if the pupil diameter measurement(s) detected by the vision screening device 304 in response to the graphical representation are within a learned or known (e.g., predetermined) threshold of the standard values (e.g., if the measurements fall within a standard range) known for patients of the same testing category, the diagnosis recommendation component 326 may generate a recommendation indicating that the patient 306 has passed the vision screening. Alternatively, if the pupil diameter measurement(s) fall outside of the standard value range, the diagnosis recommendation component 326 may generate a recommendation indicating that the patient 306 has failed the vision screening test and/or indicating that the patient 306 should receive additional screening.

The diagnosis recommendation component 326 may also generate the behavior likelihood score as described herein. For example, based on data from the patient data component 318, measurement data component 322, and/or the threshold data component 324, the diagnosis recommendation component 326 may output a score indicative of a likelihood for the patient 306 to participate in a particular type of activity. The diagnosis recommendation component may use a machine learning component 328 to perform the determination of the behavior likelihood score. For example, a machine learning model of the machine learning component 328 may be used to generate a likelihood score after being trained using eye test data, such as data from the measurement data component 322 gathered for a large number of patients, labeled with indicators of behavior exhibited by the patients, such as indications of violence, etc.

As used herein, network 308 is typically any type of wireless network or other communication network known in the art. Examples of network 308 include the Internet, an intranet, a wide area network (WAN), a local area network (LAN), and a virtual private network (VPN), cellular network connections and connections made using protocols such as 802.11a, b, g, n and/or ac. U.S. Pat. No. 9,237,846, filed Feb. 37, 2012, describes systems and methods for photo refraction ocular screening and that disclosure is hereby incorporated by reference in its entirety.

As described herein, a processor, such as processor(s) 314, can be a single processing unit or a number of processing units, and can include single or multiple computing units or multiple processing cores. The processor(s) 314 can be implemented as one or more microprocessors, microcomputers, microcontrollers, digital signal processors, central processing units, state machines, logic circuitries, and/or any devices that manipulate signals based on operational instructions. For example, the processor(s) 314 can be one or more hardware processors and/or logic circuits of any suitable type specifically programmed or configured to execute the algorithms and processes described herein. The processor(s) 314 can be configured to fetch and execute computer-readable instructions stored in the computer-readable media 330, which can program the processor(s) 314 to perform the functions described herein.

The computer-readable media 330 may can include volatile and nonvolatile memory and/or removable and non-removable media implemented in any type of technology for storage of information, such as computer-readable instructions, data structures, program modules, or other data. Such computer-readable media 330 can include, but is not limited to, RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. The computer-readable media 330 can be a type of computer-readable storage media and/or can be a tangible non-transitory media to the extent that when mentioned, non-transitory computer-readable media exclude media such as energy, carrier signals, electromagnetic waves, and signals per se.

The computer-readable media 330 can be used to store any number of functional components that are executable by the processor(s) 314. In many implementations, these functional components comprise instructions or programs that are executable by the processor(s) 314 and that, when executed, specifically configure the one or more processor(s) 314 to perform the actions attributed above to the vision screening device 304 and/or the vision screening system 310.

The network interface(s) 316 may enable wired and/or wireless communications between the components and/or devices shown in environment 300 and/or with one or more other remote systems, as well as other networked devices. For instance, at least some of the network interface(s) 316 may include a personal area network component to enable communications over one or more short-range wireless communication channels. Furthermore, at least some of the network interface(s) 316 may include a wide area network component to enable communication over a wide area network.

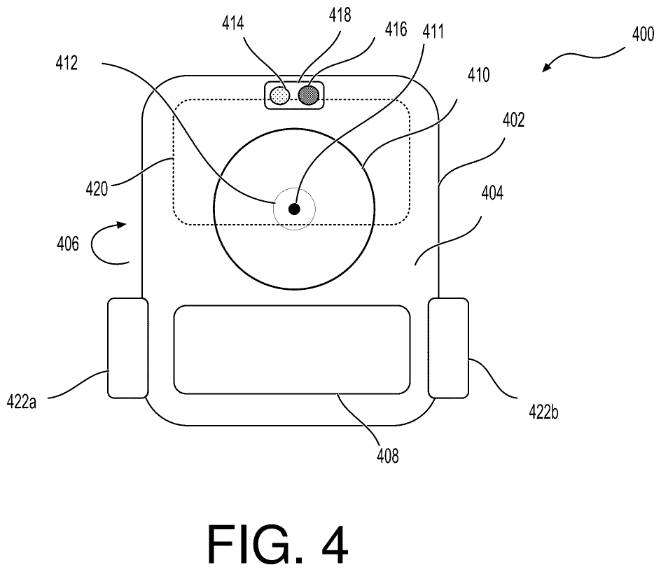

FIG. 4 illustrates an embodiment of a vision screening device 400 according to some implementations. The example vision screening device 400 may include one or more of the same components included in the vision screening device 104 of the system 100. In some additional examples, the vision screening device 400 can include different components that provide similar functions to the vision screening device 104.

The vision screening device 400 may be a tablet-like device, which may include one or more processors, computer-readable media, and network interface(s) associated therewith (not shown) in a housing 402. The housing 402 may include a front surface 404 configured to face a patient during use of the vision screening device 400, and a back surface 406, opposite the front surface 404, configured to face an operator of the vision screening device 400 (such as the user 106) during use of the vision screening device 400. The front surface 404 may include a display screen 408, radiation source(s) 410, radiation sensor(s) 412, white light source(s) 414, and/or a camera 416.

The radiation source(s) 410 may be configured to emit radiation in the infrared band and/or the near-infrared (NIR) band. For instance, the radiation source(s) 410 may comprise an arrangement of NIR LEDs configured to determine refractive error associated with one or more eyes of the patient. The NIR LEDs of the radiation source(s) 410 may be disposed radially around a central axis 411 of the vision screening device 400, with the radiation sensor(s) 412 being disposed substantially along the central axis 411. The NIR LEDs may be used to provide eccentric illumination of the eye(s) of the patient during the vision screening test(s) by aligning the central axis 411 with an optical axis of the eye(s) (e.g., for measuring refractive error using photorefraction techniques).

The vision screening device 400 may also include a white light source 414, and a camera 416 configured to captured color images and/or video of the eyes of the patient. In some examples, the white light source 414 and the camera 416 may be included in an image capture module 418. The camera 416 of the image capture module 418 may include a high-resolution lens with a narrow field of view suitable for imaging eyes in a vision screening setting. Such a lens may incorporate folded prism slim lens technology which allows for telephoto zoom while maintaining a low height profile. The optical system used in folded prism lenses bends and focuses light while it is reflected back and forth inside optical prisms, reducing the thickness of the lens and allowing for a substantially low-height form factor. Some vision screening tests may require color images of pupils and/or lenses of the eyes of the patient to determine the presence of diseases and/or abnormalities. In some examples, the camera 416 may be equipped with a high-resolution zoom capability to enable the capture of close-up images of the eyes of the patient from which the pupils and/or lenses of the eyes can be localized. In other examples, the camera 416 may use a fixed focal length lens with placement of the eyes being adjusted to achieve an in-focus image. The white light source 414, which is more commonly referred to as a flash, may include one or more visible light LEDs of adjustable intensity. The intensity level of the white light source 414 may be controlled by the one or more processors of the vision screening device 400. The one or more processors of the vision screening device 400 may also synchronize timing of activation of the white light source(s) 414 with the capture of an image by the camera 416.

The vision screening device 400 may also include a display screen 420 disposed on the back surface 406 of the housing 402 that substantially faces the operator (e.g., the operator 102), during operation of the vision screening device 400. The display screen 420, which may be touch-sensitive to receive inputs from the operator, may display a graphical user interface configured to display information to the operator and/or receive input from the operator during a vision screening test. For example, the display screen 420 may be used by the operator to enter information regarding the patient, or the vision screening test(s) being administered. Further, the display screen 420 may be configured to display information to the operator regarding the vision screening test being administered (e.g., parameter settings, progress of screening, options for transmitting data from the vision screening device 400, one or more measurements, and/or images or visualizations generated during the vision screening, etc.). The display screens 408, 420 may comprise, for example, a liquid crystal display (LCD) or active matrix organic light emitting display (AMOLED).

In some examples, the vision screening device 400 may include hand grips 422a and 422b for holding the vision screening device 400 with stability during the vision screening tests. As discussed herein, FIG. 4 depicts an exemplary vision screening device 400 that includes components for administering one or more vision screening test(s) to a patient. The vision screening device 400 is intended to perform an entire vision screening which may include multiple, different vision screening tests including screening for multiple diseases, abnormalities and conditions of the eyes of the patient. Accordingly, the vision screening device 400 may be used for determining the behavior likelihood score discussed herein. The vision screening device 400, as shown, has the additional features of being light weight enough to be hand-held, by using the hand grips 422a and 422b for the right and left hand of the operator respectively as an example, allowing for portability and ease-of-use in patients as young as newborn. The vision screening device 400 provides the radiation sources and image capture sensors needed for NIR imaging, as well as color imaging under white light illumination as required for one or more vision screening test(s), in a compact and substantially planar arrangement, enabling the light weight and portable form factor of the vision screening device 400.

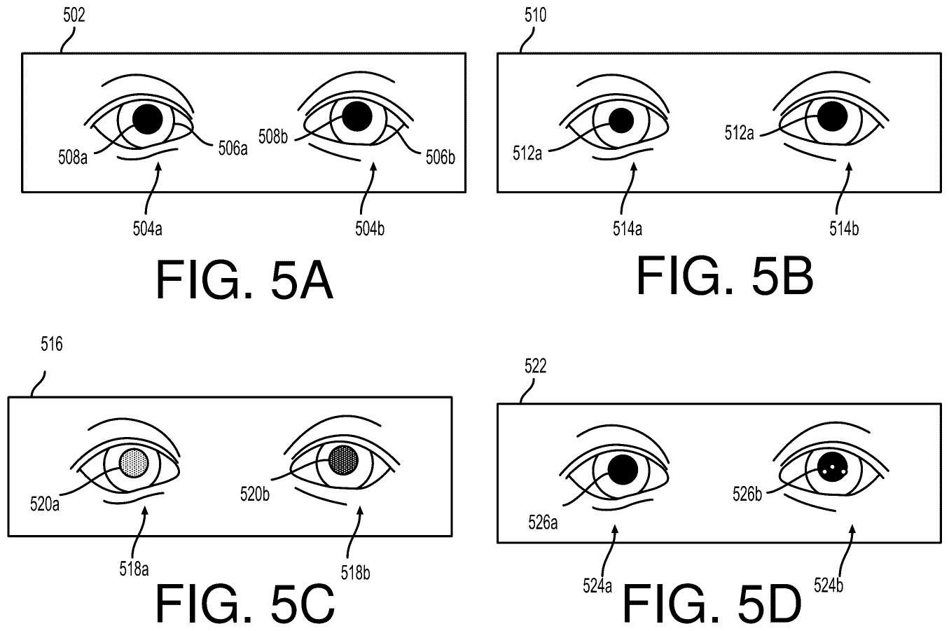

FIGS. 5A-5D illustrate images of eyes captured by the radiation sensor(s) or the camera of the vision screening device 104. Various abnormalities and/or diseases of the eye(s) that may be detected utilizing analysis of image data captured by the vision screening device 104, are discussed herein with reference to FIGS. 5A-5D. The abnormalities and/or conditions associated with the eyes may include characteristics of the eyes and/or pupils. The characteristics of the eye and/or pupil that may be evaluated include ptosis, abnormal pupil size, nonreactivity of the pupil to a light challenge, nystagmus, non-convergence, hippus, redness or swelling at or around the eyes, and other such visibly detectable characteristics. In some examples, the vision screening device may detect conditions indicative of substance abuse, impairment, central nervous system issues, epilepsy, tumors, and other such conditions. In some examples, nystagmus may refer to repetitive, uncontrolled movements of the eyes. Nystagmus may be identifiable based on eye jitters or sudden jumps by the eyes as they track an item. In some examples, nystagmus may be identified when the eyes exhibit a linear jump greater than ten percent of a moving distance to track an item. Hippus may refer to a restless mobility of the pupil, a tendency for the pupil size to fluctuate when it should otherwise be stable. For example, a pupil of an individual may pulsate or change in diameter over a short period of time. In some examples, the pupil diameter may be measured over time in response to an illumination stimuli. The response may be compared against historical examples (e.g., for the particular patient), from the right eye to the left eye, or against other benchmarks. The response rate of the pupil to the stimuli (illumination) may correlate to particular neurological conditions and/or impairments. In some examples, the vision screening device may know a light level within the environment (e.g., using a light meter) and the system may use the light level within the environment to identify a mismatch between the pupil size and the level of illumination in the environment (e.g., are the pupils overly dilated or too small given the lighting conditions?). In some examples, the system may also access electronic medical record data for the patient to identify a medication and/or an eye prescription that may impact the performance of the eyes and/or pupils.

FIG. 5A illustrates an image 502 of the eyes of a patient with normal eye health and no detectable disease conditions or abnormalities and unaffected by substances that may otherwise affect the behavior of the patient. The image 502 includes the right eye 504a and the left eye 504b of the patient. As shown, the iris 506a and pupil 508a of the right eye 504a appear substantially similar to the corresponding iris 506b and pupil 508b of the left eye 504b in patients exhibiting normal eye health. The data analysis and visualization component 236 may process the captured images to determine location of pupils of the eye(s), and generate images of the pupils (e.g., pupil images). Since the pupils allow radiation to enter the interior of the eye and reflected radiation to return out of the eye after interaction with different layers of the eye, the pupil images capture the appearance of layers of the eye(s), such as cornea, lenses, aqueous and vitreous humors, and retina which are illuminated by the radiation impinging on the eye. U.S. patent application Ser. No. 17/347,079, filed on Jun. 14, 2021, the entire disclosure of which is incorporated herein by reference, describes example systems and methods for detecting pupil images captured under different illumination patterns generated by near-infrared (NIR) radiation sources for determining refractive error based on photorefraction.

FIG. 5B illustrates an image 510 of a disease condition that may be detected by comparing a pupil image 512a of one eye 514a with a pupil image 512b of the other eye 514b. The image 510 may include a grayscale image captured by the radiation sensor(s) under NIR illumination and/or a color image captured by a camera, and illustrates an example of a difference in size of pupils in the images of the left and right eye as a result of one or more conditions associated with the patient. The data analysis and visualization component 236 may compare pupil images of the left and the right eyes to determine a difference in pupil size or other pupil characteristics between the eyes. The computed difference may be compared with threshold(s) and/or ranges in standard test data corresponding to normal eyes to determine if an abnormality is present.

FIG. 5C illustrates an image 516 captured under white light illumination by the camera(s) of the vision screening device 104. Since the pupil images 520a, 520b of the image 516 are generated from white light reflected back from the retina of the eyes, and through the cornea, diseases and abnormalities of the retina and cornea may be visible in such an image. For example, since the retina is highly vascular, the reflected light may appear to be of an orange-red color in a healthy eye, but may appear to be white or yellowish in an eye with a retinal or corneal tumor. While there may be variations in the appearance of the color of the pupil images 520a, 520b due to different pigmentations of the retina among patients of different ethnicities, comparisons between the two pupil images 520a and 520b of the same patient may reliably produce differences in color values when the disease or abnormality is present in only one of the two eyes. Additionally, the use of the color data may enable determinations of measurements for redness of the eyes and/or around the eyes, including at the eyelids, that may be associated with certain behavioral triggers, such as substance use.

FIG. 5D further illustrates an image 522 of the eyes 524a, 524b, including pupil images 526a, 526b. The image 522 may be grayscale image captured by the radiation sensor(s) under NIR illumination or a color image captured by the camera(s) under white light illumination. As shown, using the glints or reflections off of the pupils 526a and 526b, the movement and/or gaze direction of the patient may be observed. The movement and/or direction of gaze may be used to identify rapid eye movements, differences in eye movements, misalignments, and other such differences and/or abnormalities that may be due to impairment or conditions of the eyes of the patient.

In some examples, the data analysis and visualization component 236 may determine some conditions of the eyes by evaluating each pupil image, taken individually, for uniformity of characteristics in the pupil image. The characteristics may include color, brightness, texture, etc. For example, the data analysis and visualization component 236 may determine standard deviation (or variance) in the characteristic within the pupil image, and if the standard deviation is higher than a threshold, or outside a range expected in a healthy eye, an abnormal condition may be determined.

Though FIGS. 5A-4D show examples of some conditions of the eyes that may be determined by the techniques discussed herein, it should be understood that additional conditions may also be determined. In addition, the data analysis and visualization component 236 may analyze a color image under white light illumination, a grayscale image under NIR illumination, and/or a composite image, to determine a condition of the eyes. For example, a presence of a cataract in the eye(s) may be determined based on the composite image, whereas a presence of a blastoma may be primarily determined based on the color image. In some examples, the color image and/or the grayscale image may be extracted from a color and/or grayscale video of the eyes e.g., one or more frames of the video.

As discussed herein, FIGS. 5A-4D illustrates processing of images captured by the radiation sensor(s) and/or the camera(s) that may be performed by the data analysis and visualization component of the vision screening device in order to determine differences between pupil images indicative of disease conditions and/or abnormalities of the eye(s) of the patient. Other examples of processing tailored for detecting specific disease conditions and abnormalities are also envisioned. For example, images captured under different wavelengths of radiation may be used for detecting signature differences in grayscale or color values or structure of images indicative of specific disease conditions.

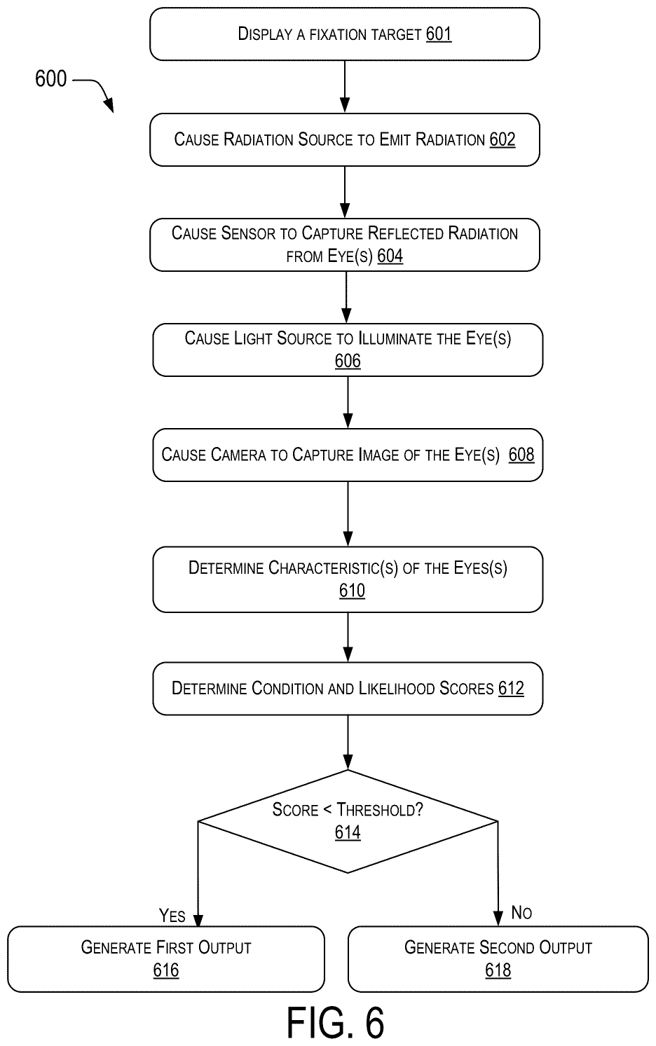

FIG. 6 provides a flow diagram illustrating an example method for vision screening, as described herein. The method in FIG. 6 is illustrated as collections of blocks in a logical flow graph, which represents sequences of operations that can be implemented in hardware, software, or a combination thereof. In the context of software, the blocks represent computer-executable instructions stored on one or more computer-readable storage media that, when executed by processor(s), perform the recited operations. Generally, computer-executable instructions include routines, programs, objects, components, data structures, and the like that perform particular functions or implement particular abstract data types. The order in which the operations are described is not intended to be construed as a limitation, and any number of the described blocks can be combined in any order and/or in parallel to implement the method illustrated in FIG. 6. In some embodiments, one or more blocks of the method illustrated in FIG. 6 can be omitted entirely.

The operations described below with respect to the method illustrated in FIG. 6 can be performed by any of the devices or systems described herein, and/or by various components thereof. Unless otherwise specified, and for ease of description, the method illustrated in FIG. 6 will be described below with reference to the system 200 shown in FIG. 2.

With reference to the example method 600 illustrated in FIG. 6, at operation 601, the vision screening device, or another device associated with the system, may display a fixation target for the user to view. The fixation target may be presented on a display of the vision screening device facing the patient or may be displayed using an alternative display separate from the vision screening device. The fixation target may include a statis and/or dynamic target that the patient is directed to focus on, follow, or otherwise view while the vision screening device captures data regarding the eyes of the patient.

At operation 602, the image capture control component 234 and/or one or more processors associated therewith may cause a radiation source to emit radiation (e.g., near-infrared (NIR) radiation). For example, the radiation source may comprise NIR LEDs of the radiation source(s) 214 of the vision screening device 204, configured to emit NIR radiation during a period of time corresponding at least in part to the administration of a vision screening test indicated for the patient by the patient screening component 232. In some examples, the image capture control component 234 may cause radiation of different wavelengths to be emitted e.g., a first radiation source may emit radiation of a first wavelength, and a second radiation source may emit radiation of a second wavelength. The image capture control component 234 may activate LEDs of the radiation source(s) 214 individually or in groups to produce radiation impinging on the eyes at different angles relative to the optical axis of the eye. For example, the image capture control component 234 may set a pattern of activation of the NIR LEDs along various axes. In addition, the image capture control component 234 may activate different wavelengths of radiation combined with different angles of incidence on the eyes. In some examples, the arrangement of the pattern of activation of the NIR LEDs allows for different illumination patterns to be presented to the eye(s) of the patient 206, and refractive error of the eye(s) may be accurately measured based on images captured under the illumination pattern selected. Additional details regarding illumination patterns used in examination protocols for determining refractive error can be found in U.S. Pat. No. 9,237,846, referred to above and incorporated herein by reference.

At operation 604, the image capture control component 234 may cause a sensor of the vision screening device (e.g., radiation sensor(s) 216 of the vision screening device 204), to capture radiation reflected by the eye(s) of the patient under illumination by the radiation source(s) 214. The image capture control component 234 may receive data indicative of the radiation captured by the radiation sensor(s) 216. The data may include grayscale image(s) and or video of the eye(s) illuminated by radiation from different angles as described above at operation 602. For example, the image capture control component 234 may cause a sensor to capture a first image under illumination from a first set of NIR LEDs, and a second image under illumination from a second set of NIR LEDs. In some examples, near-infrared may be a first wavelength band emitted by a first radiation source(s), and the image capture control component 234 may further activate a second radiation source(s) emitting radiation in a second wavelength band, and cause a sensor to capture a third image under illumination from the second radiation source. In addition, the image capture control component 234 may cause the sensor(s) to capture images of both eyes simultaneously, or one eye at a time. For example, the image capture control component 234 may change the activation of the illumination source(s) (e.g., activate different LEDs of the radiation source(s) 214, 218) after capturing an image of the left eye and before capturing an image of the right eye, so that both the left and right eye are illuminated from a same angle relative to the eye during the image capture. In some examples, the image capture control component 234 may cause the sensor to capture multiple images of the eyes while the patient 206 is directed to look in different directions e.g., the patient's gaze direction may be to the left, right, up and/or down with respect to an optical axis of the vision screening device 204. In some examples, the sensor may capture reflected radiation during and/or after the radiation source is emitting radiation, for example to gather data for how the eye responds to changes in lighting.

At operation 606, the image capture control component 234 and/or one or more processors associated therewith may cause a white light source (e.g., white light source(s) 218 of the vision screening device 204), to emit white light to illuminate the patient during a period of time after the operation 602 and 604 are completed, and during at least a part of the administration of the vision screening test. Similar to the radiation source(s) described at operation 602, individual white light sources of the white light source(s) 218 may also be activated to illuminate the eye from different angles relative to the optical axis. In some examples, the illumination from the white light source(s) 218 may be coaxial or near-coaxial with the optical axis e.g., the angle may be substantially zero degrees. In some examples, multiple images of the eyes may be captured corresponding to different gaze directions of the patient 206 as described above e.g., a first color image of the eye(s) may be captured corresponding to a first gaze direction of the patient and a second color image of the eye(s) may be captured corresponding to a second gaze direction of the patient. The image capture control component 234 may store, as metadata associated with an image, a time of capture and the angle of illumination and/or the gaze direction of the patient at the time of capture of the image.

At operation 608, the image capture control component 234 may cause a camera (e.g., the camera 220 of the vision screening device 204), to capture color image(s) of the eye(s) of the patient while under white light illumination. In some examples, the image capture control component 234 may also cause the camera to capture video data. For example, video data may be captured during a first period of time before onset of white light illumination, and continue during a second period of time after commencement of the white light illumination. For example, the video data may be useful for determining a reaction of the patient's pupils (e.g., size of the pupils) and/or an adjustment of the pupils to varying levels of illumination and/or a sharp change in illumination (e.g., caused by onset of the white light illumination). The image capture control component 234 may store the color image(s) and/or video in a database for review by a clinician. In addition, the image capture control component 234 may cause the camera to capture a color image of the patient's face and store the image in the patient data 240 as a photo identifier of the patient. In some examples, the camera may capture the image data during and/or after the light source is emitting light, for example to gather data for how the eye responds to changes in lighting.

At operation 610, the data analysis and visualization component 236 may determine a characteristic of the eye(s). The characteristic may relate to the eyes, the pupils, the eyelids, or other such features visible to the sensor and/or camera. The data analysis and visualization component 236 may detect pupil images corresponding to the pupils of the eye(s) in the grayscale image(s) and the color image(s), and align the grayscale and color pupil images so that structures of the eyes overlap. The data analysis and visualization component 236 may also annotate (e.g., using graphics and/or pseudo-color values) some portions of the composite image to indicate areas of interest, for example. The characteristic(s) of the eye(s) may include dilation of the pupils, response rates of the pupils to a stimulus (e.g., a light), differences between the two eye, redness or swelling around the eyes (e.g., at the eyelids), or other such characteristics that may be measured and/or observed by the systems described herein.

At operation 612, the output generation component 238 may determine one or more condition and/or likelihood scores. Based at least in part on the analysis of the captured images from the vision screening device during the one or more vision tests, the vision screening device (and/or a computing device associated with the device) may generate an output including a recommendation, prediction, evaluation, or other indication of the status of the patient and/or a prediction of certain behaviors that may be associated with a particular predicted condition such as outbursts, flight risk, violent behavior, etc. For example, the device may detect if a patient is impaired due to drug use and indicate a violence risk score based on the captured images. Notably, the vision screening device may provide such indications earlier than a blood test may return a result indicative of such impairment or altered status and therefore the caregivers may be prepared to allocate resources as necessary before an incident occurs. In some examples, the vision screening device and system described herein may provide an indication of a condition, a likelihood of particular behavior, and may also identify a particular protocol or intervention procedure based on the condition and/or likelihood of behavior. The protocol or intervention procedure may include allocating resources within a facility, specifying a particular treatment protocol (e.g., removing a patient from a noisy waiting area if they have a high likelihood of having a condition that may cause them to become overly stimulated or agitated in a busy environment), or particular type of intervention or preparation for intervention (e.g., identifying when treatment for a potential overdose or substance abuse is likely and preparing materials for a potential intervention). The vision screening device may be used as part of the routine physical assessment at triage on a patient when they enter an emergency department or other caregiving location.

Based on a comparison with a threshold and/or range described above, the data analysis and visualization component 236 may generate a behavior likelihood score for the patient 206 in addition to vision test results. For example, a score with respect to behavior associated with violence may be displayed as a numerical value on a scale of zero to one hundred, or may be provided as a rating of the prediction for violence being low, medium, high, or extreme, with thresholds for the behavior likelihood score associated with each of the ranges.

In examples, the data analysis and visualization component 236 may utilize one or more machine learning techniques to generate a behavior likelihood score in addition to diagnosis of specific diseases and/or types of abnormalities. For example, machine learning (ML) models may be trained with normal images of eyes, and images of eyes labeled with data indicative of behavior exhibited by the individual (e.g., as stored in patient medical record data) as well as labels exhibiting various disease conditions and abnormalities. The trained ML model(s) may then generate an output indicating the behavior likelihood score as well as a disease or abnormality diagnosis when provided, as input, an image of the eye captured during the vision screening of the patient 206. In such examples, the data analysis and visualization component 236 may directly generate the output by providing an image of the eye as input to the trained ML model(s), without computing differences between pupil images or applying comparisons with a threshold and/or range. In some examples, the images may be used to determine one or more characteristics, such as pupil characteristics over the duration of the test that may then be provided, either with or in place of the image data, to the trained ML model(s). In some examples, a plurality of trained ML model(s) may be used, each ML model being trained to determine behavior likelihood scores for various behavioral profiles, as may be determined by a caregiver facility. The data analysis and visualization component 236 may provide an image of the eye as input to each ML model of the plurality of trained ML model(s) for determining the behavior likelihood scores. In examples, the ML models may be neural networks, including convolutional neural networks (CNNs). In other examples, the ML models can also include regression algorithms, decision tree algorithms, Bayesian classification algorithms, clustering algorithms, support vector machines (SVMs) and the like.

The output generation component 238 may be configured to receive, access, and/or analyze data from the data analysis and visualization component 236, and generate the output. For example, the output generation component 238 may utilize the behavior likelihood scores of the data analysis and visualization component 236 to generate a recommendation in the output. The recommendation may indicate a course of action for treating the patient 206, for allocating resources at the facility, for example to prepare to handle a particular behavior that is predicted to be forthcoming based on the behavior likelihood scores, and other such outputs. The output may be presented to the operator of the device via an interface of the device (e.g., on the display screen of the vision screening device). In examples, the operator display screen may not visible to the patient, e.g., the operator display screen may be facing in a direction opposite the patient

At operation 614, the output generation component 238 may compare the behavior likelihood score and threshold value(s) and/or range(s) to determine an output indicative of a probability that the patient 206 will engage in a particular behavior, which may include a diagnosis or recommendation. For example, if the score is less than the threshold value (Operation 614—Yes), the output generation component 238 may generate a first output associated with the patient at operation 616, and if the difference is equal to or higher than the threshold value (Operation 614—No), the output generation component 238 may generate a second output at operation 618. The threshold value(s) and/or range(s) may be predetermined and available as a part of standard data, which may be stored in the screening database 244 or the computer-readable media 230, 248. The standard data may include different threshold(s) and range(s) for each type of the behavior scores that may be defined.

At operation 616, the output generation component 238 may generate the first output as described above (Operation 614—Yes). The first output may correspond to an indication that the likelihood of particular behavior (e.g., violence) is low or unexpected. At operation 618, the output generation component 238 may generate the second output (when Operation 614-No). The second output may correspond to an indication of that the patient has a high likelihood of the particular behavior. In the event that the score is over the threshold, the output generation component 238 may generate a recommendation for treatment of the patient and/or for distributing resources at the facility to accommodate the expected behavior (e.g., additional staff or security, additional equipment or tools available, etc.) such that the caregivers may be prepared in the event that the patient 206 does exhibit the predicted behavior. In this manner, they may be prepared for the behavior and react quickly and responsively in an equipped manner, rather than being unprepared for such behavior.

As discussed, the example method 600 may be performed by the components of the vision screening device 204 executed by the processor(s) 228 of the vision screening device 204. The example method 600 illustrates operations performed during at least a part of a vision screening test administered to a patient (e.g., the patient 206) to determine behavior likelihood scores of the patient based on images of the eye(s) captured under illumination. In alternative examples, some or all of the operations of example method 600 may be executed by processor(s) 246 of a vision screening system 210 that is connected to the vision screening device 204 via network 208.

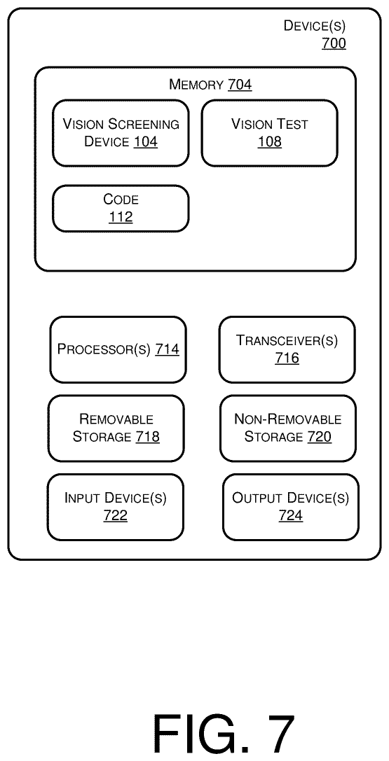

FIG. 7 illustrates a device 700 configured to enable and/or perform the some or all of the functionality discussed herein. Further, the device(s) 700 can be implemented as one or more server computers 702, a network element on a dedicated hardware, as a software instance running on a dedicated hardware, or as a virtualized function instantiated on an appropriate platform, such as a cloud infrastructure, and the like. It is to be understood in the context of this disclosure that the device(s) 700 can be implemented as a single device or as a plurality of devices with components and data distributed among them.

As illustrated, the device(s) 700 comprise a memory 704. In various embodiments, the memory 704 is volatile (including a component such as Random Access Memory (RAM)), nonvolatile (including a component such as Read Only Memory (ROM), flash memory, etc.) or some combination of the two.

The memory 704 may include various components, such as at least of the vision screening device 104, the vision test 108, the code 112, or other such information. Any of the vision screening device 104, the vision test 108, or the code 112 can include methods, threads, processes, applications, or any other sort of executable instructions. The vision screening device 104, the vision test 108, or the code 112.

The memory 704 may include various instructions (e.g., instructions in the vision screening device 104, the vision test 108, or the code 112), which can be executed by at least one processor(s) 714 to perform operations. In some embodiments, the processor(s) 714 includes a Central Processing Unit (CPU), a Graphics Processing Unit (GPU), or both CPU and GPU, or other processing unit or component known in the art.

The device(s) 700 can also include additional data storage devices (removable and/or non-removable) such as, for example, magnetic disks, optical disks, or tape. Such additional storage is illustrated in FIG. 7 by removable storage 718 and non-removable storage 720. Tangible computer-readable media can include volatile and nonvolatile, removable and non-removable media implemented in any method or technology for storage of information, such as computer readable instructions, data structures, program modules, or other data. The memory 704, removable storage 718, and non-removable storage 720 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device(s) 700. Any such tangible computer-readable media can be part of the device(s) 700.

The device(s) 700 also can include input device(s) 722, such as a keypad, a cursor control, a touch-sensitive display, voice input device, etc., and output device(s) 724 such as a display, speakers, printers, etc. These devices are well known in the art and need not be discussed at length here. In particular implementations, a user can provide input to the device(s) 700 via a user interface associated with the input device(s) 722 and/or the output device(s) 724.

As illustrated in FIG. 7, the device(s) 700 can also include one or more wired or wireless transceiver(s) 716. For example, the transceiver(s) 716 can include a Network Interface Card (NIC), a network adapter, a LAN adapter, or a physical, virtual, or logical address to connect to the various base stations or networks contemplated herein, for example, or the various user devices and servers. To increase throughput when exchanging wireless data, the transceiver(s) 716 can utilize Multiple-Input/Multiple-Output (MIMO) technology. The transceiver(s) 716 can include any sort of wireless transceivers capable of engaging in wireless, Radio Frequency (RF) communication. The transceiver(s) 716 can also include other wireless modems, such as a modem for engaging in Wi-Fi, WiMAX, Bluetooth, or infrared communication.

In some implementations, the transceiver(s) 716 can be used to communicate between various functions, components, modules, or the like, that are comprised in the device(s) 700. For instance, the transceivers 716 may facilitate communications between the vision screening device 104 and other devices storing the vision test 108, the code 112, or other such information.

The foregoing is merely illustrative of the principles of this disclosure and various modifications can be made by those skilled in the art without departing from the scope of this disclosure. The above described examples are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

As a further example, variations of apparatus or process limitations (e.g., dimensions, configurations, components, process step order, etc.) can be made to further optimize the provided structures, devices and methods, as shown and described herein. In any event, the structures and devices, as well as the associated methods, described herein have many applications. Therefore, the disclosed subject matter should not be limited to any single example described herein, but rather should be construed in breadth and scope in accordance with the appended claims.

What is claimed is:

1. A vision screening device, comprising:
a sensor configured to capture sensor data associated with an eye of a patient;
a processor operably connected to the sensor; and
a non-transitory memory storing instructions that, when executed by the processor, cause the processor to perform operations comprising:
causing the sensor to capture the sensor data during a first period of time;
determining a characteristic of the eye of the patient during the first period of time based on the sensor data;
determining a likelihood score based on the characteristic of the eye, the likelihood score indicative of a probability associated with one or more condition predictions or one or more behavior predictions for the patient; and
generating, based at least in part on the likelihood score, an output indicative of a behavior predicted for the patient.

2. The vision screening device of claim 1, wherein determining the likelihood score comprises inputting the characteristic of the eye into a machine learning model trained using eye characteristic data labeled with observed behaviors.

3. The vision screening device of claim 1, further comprising:
determining, in response to the likelihood score exceeding a threshold, one or more treatment protocols based on the one or more condition predictions or the one or more behavior predictions; and
conveying a signal to a system of a facility associated with the vision screening device to cause one or more actions described in the one or more treatment protocols.

4. The vision screening device of claim 1, further comprising a display unit disposed on a first side of the vision screening device and configured to display the output to an operator of the vision screening device,
wherein the sensor is disposed on a second side of the vision screening device, opposite the first side.

5. The vision screening device of claim 1, further comprising:
a radiation source comprising an array of light emitting diodes (LEDs) configured to illuminate the eye of the patient from a first angle, and a second angle different from the first angle, relative to an optical axis associated with the eye of the patient, and wherein:
the sensor comprises a camera; and
the radiation source is controllably illuminated during the first period of time.

6. The vision screening device of claim 1, wherein determining the characteristic of the eye comprises:
conveying the sensor data to a computing system communicably coupled with the vision screening device; and
receiving the characteristic of the eye from the computing system, and wherein determining the likelihood score comprises receiving the likelihood score from the computing system in response to conveying the sensor data to the computing system.

7. The vision screening device of claim 1, further comprising:
determining, in response to the likelihood score exceeding a threshold, one or more resources of a facility associated with the vision screening device; and
conveying a signal to cause the one or more resources to be re-allocated at the facility based on the likelihood score.

8. A method, comprising:
capturing, using a vision screening device, image data of an eye of a patient during a first period of time;
determining a characteristic of the eye of the patient during the first period of time based on the image data;
determining a likelihood score based on the characteristic of the eye, the likelihood score indicative of a probability associated with one or more condition predictions or one or more behavior predictions for the patient; and
generating, based at least in part on the likelihood score, an output indicative of a behavior predicted for the patient.

9. The method of claim 8, wherein determining the likelihood score comprises inputting the characteristic of the eye into a machine learning model trained using eye characteristic data labeled with observed behaviors.

10. The method of claim 9, wherein the eye characteristic data is labeled with observed behavior data from patient medical record data.

11. The method of claim 8, wherein determining the characteristic of the eye comprises determining a characteristic of a pupil of the eye during the first period of time.

12. The method of claim 11, wherein the characteristic of the pupil comprises at least one of:
a pupil response rate;
a pupil dilation size; or
a pupil motion indication.

13. The method of claim 8, wherein determining the characteristic of the eye and determining the likelihood score comprises:
providing, as input, the image data to a trained machine learning model; and
receiving, from the trained machine learning model, the likelihood score.

14. The method of claim 8, further comprising:
determining, in response to the likelihood score exceeding a threshold, one or more resources of a facility associated with the vision screening device; and
conveying a signal to cause the one or more resources to be re-allocated at the facility based on the likelihood score.

15. A system, comprising:
memory;
a processor; and
computer-executable instructions stored in the memory and executable by the processor to perform operations comprising:
causing a sensor to capture sensor data of an eye of a patient during a first period of time;
determining a characteristic of the eye of the patient during the first period of time based on the sensor data;
determining a likelihood score based on the characteristic of the eye, the likelihood score indicative of a probability associated with one or more conditions predictions or one or more behavior predictions for the patient; and
generating, based at least in part on the likelihood score, an output indicative of a behavior predicted for the patient.

16. The system of claim 15, wherein determining the likelihood score comprises inputting the characteristic of the eye into a machine learning model trained using eye characteristic data labeled with observed behaviors.

17. The system of claim 16, wherein the eye characteristic data is labeled with observed behavior data from patient medical record data.

18. The system of claim 15, wherein determining the characteristic of the eye comprises determining a characteristic of a pupil of the eye during the first period of time.

19. The system of claim 15, wherein the operations comprise additional operations comprising:
determining, in response to the likelihood score exceeding a threshold, one or more resources of a facility associated with the system; and
conveying a signal to cause the one or more resources to be re-allocated at the facility based on the likelihood score.

20. The system of claim 15, wherein determining the characteristic of the eye and determining the likelihood score comprises:
providing, as input, the sensor data to a trained machine learning model; and
receiving, from the trained machine learning model, the likelihood score.
